(19) United States
(12) Patent Application Publication
(10) Pub. No.: US 2021/0347868 A1
(43) Pub. Date: Nov. 11, 2021

(54) ANTI-SYNUCLEIN ANTIBODIES

(71) Applicants: Gabriel PASCUAL, San Diego, CA (US); Adrian APETRI, Noordwijkerhout (NL); Xinji LI, San Diego, CA (US)

(72) Inventors: Gabriel PASCUAL, San Diego, CA (US); Adrian APETRI, Noordwijkerhout (NL); Xinji LI, San Diego, CA (US)

(21) Appl. No.: 17/283,229

(22) PCT Filed: Oct. 17, 2019
(86) PCT No.: PCT/EP2019/078146

§ 371 Date: Apr. 6, 2021

Related U.S. Application Data
(60) Provisional application No. 62/747,870, filed on Oct. 19, 2018.

(51) Int. Cl. C07K 16/18; A61K 47/68; G01N 33/577; G01N 33/68

(52) U.S. Cl. CPC C07K 16/18; A61K 47/6843; G01N 33/577; G01N 33/6896; C07K 2317/565; C07K 2317/92; C07K 2317/76; G01N 2800/2814; G01N 2800/2835; C07K 2317/21; C07K 2317/34

(57) ABSTRACT

Anti-alpha-synuclein antibodies and antigen-binding fragments thereof are described. Also described are nucleic acids encoding the antibodies, compositions comprising the antibodies, and methods of producing the antibodies and using the antibodies for treating or preventing diseases characterized by Lewy bodies or alpha-synuclein aggregation.

### FIELD OF THE INVENTION

This invention relates to monoclonal anti-alpha-synuclein antibodies, nucleic acids and expression vectors encoding the antibodies, recombinant cells containing the vectors, and compositions comprising the antibodies. Methods of making the antibodies, and methods of using the antibodies to diagnose and treat diseases characterized by Lewy bodies or alpha-synuclein aggregation are also provided.

### BACKGROUND OF THE INVENTION

Parkinson's disease (PD) is the second most common neurodegenerative disorder with symptoms including tremor, rigidity, slowness of movement, and impaired balance and coordination. About 50,000 people are diagnosed with PD in the U.S. each year and about half a million people have the disease (NIH Fact sheets, Parkinson's Disease).

The neuropathological hallmark of PD is Lewy bodies and Lewy neurites, abnormal protein aggregates that are primarily comprised of alpha-synuclein filaments (Goedert et al., 2013). PD, PD with dementia, and dementia with Lewy bodies are all Lewy body diseases that affect 5 million people worldwide (Lashuel et al., 2013). Multiple system atrophy (MSA) (Spillantini et al., 1998) and some lysosomal-storage diseases, such as Gaucher's disease (Shachar et al., 2011), also exhibit alpha-synuclein aggregation. Moreover, 50% of Alzheimer's disease (AD) cases show Lewy Body pathology (Ditter and Mirra, 1987). Additionally, alpha-synuclein regulates aggregation of amyloid-O (Bachhuber et al., 2015) and tau (Guo et al., 2013), two proteins associated with neuropathological hallmark of AD.

Analysis of Lewy body pathology suggests a progressive spreading of alpha-synuclein aggregates with disease progression or clinical progression of PD, which suggests that spreading of alpha-synuclein aggregates is the driver of the disease pathology (Braak and Del Tredici, 2008).

Alpha-synuclein is an intrinsically disordered protein of 140 amino acids, primarily composed of three regions, i.e. an amino terminus responsible for membrane interaction; a disordered acidic carboxyl-terminal tail, and the hydrophobic motif (amino acid residues 65-90); known as non-amyloid-O component of AD amyloid plaques (NAC), that is critical for aggregation of alpha-synuclein. Point mutations (A30P, E46K, H50Q, G51D, A53E and A53T) of alpha-synuclein protein and increased dosage of SNCA, the gene encoding alpha-synuclein, are associated with the familial form of PD (Lashuel et al., 2013; Wong and Krainc, 2017). Moreover, genome-wide association study (GWAS) identified SNCA as one of the most important genetic risk factors for idiopathic PD (Goedert et al., 2013).

Passive and active immunizations against alpha-synuclein have been analyzed in mice by targeting alpha-synuclein (Games et al., 2014; Masliah et al., 2005; Masliah et al., 2011; Spencer et al., 2017; Tran et al., 2014). For example, Masliah et al. actively immunized a transgenic mouse model with recombinant alpha-synuclein protein (Masliah et al., 2005). The mice produced antibodies against alpha-synuclein protein leading to a significant amelioration of the accumulation of alpha-synuclein protein (Masliah et al., 2005). Passive immunization with monoclonal antibodies against the C-terminus of alpha-synuclein improves the behavioral deficits associated with alpha-synuclein deposition in synucleinopathy mouse models (Bae et al., 2012; Games et al., 2014; Masliah et al., 2011). In mice injected with synthetic alpha-synuclein fibrils, injection of an antibody against the N-terminus of α-synuclein improved Lewy body pathology and reduced neurodegeneration (Tran et al., 2014).

Clinical trials of immunotherapy directly targeting α-synuclein include active vaccines PD01A and PD03A (Schneeberger et al., 2016) or passive immunotherapy with antibodies PRX002 (Schenk et al., 2017) and BIIB054 (Weihofen et al., 2016).

The incidence of PD increases with age and the cost to society increases without an effective method to diagnose, prevent and treat the disease. Currently, PD is diagnosed when most of dopamine nerve cells are already lost and none of the methods of treatment can significantly slow the underlying neurodegeneration (NIH Fact sheets, Parkinson's Disease). Discovery of new diagnostic methods and therapeutics for PD and other Lewy body diseases is critical.

### BRIEF SUMMARY OF THE INVENTION

In one general aspect, the invention relates to isolated monoclonal antibodies (mAbs) or antigen-binding fragments thereof that bind human alpha-synuclein.

Provided are isolated monoclonal antibodies or antigen-binding fragments thereof comprising a heavy chain complementarity determining region 1 (HCDR1), HCDR2, HCDR3, a light chain complementarity determining region 1 (LCDR1), LCDR2, and LCDR3, having the polypeptide sequences of:
(a) SEQ ID NOs: 7, 8, 9, 16, 17, and 18, respectively;
(b) SEQ ID NOs: 10, 11, 12, 19, 20, and 21, respectively; or
(c) SEQ ID NOs: 13, 14, 15, 22, 23, and 24, respectively;
   wherein the antibody or antigen-binding fragment thereof specifically binds alpha-synuclein, preferably human alpha-synuclein.

In certain embodiments, the isolated monoclonal antibody or antigen-binding fragment thereof comprises a heavy chain variable region having a polypeptide sequence at least 95% identical to SEQ ID NO: 1, 3, or 5, or a light chain variable region having a polypeptide sequence at least 95% identical to SEQ ID NO: 2, 4, or 6.

In certain embodiments, the isolated monoclonal antibody or antigen-binding fragment thereof comprises:
(a) a heavy chain variable region having the polypeptide sequence of SEQ ID NO:1, and a light chain variable region having the polypeptide sequence of SEQ ID NO:2;
(b) a heavy chain variable region having the polypeptide sequence of SEQ ID NO:3, and a light chain variable region having the polypeptide sequence of SEQ ID NO:4; or
(c) a heavy chain variable region having the polypeptide sequence of SEQ ID NO:5, and a light chain variable region having the polypeptide sequence of SEQ ID NO:6.

Also provided are isolated monoclonal antibodies or antigen-binding fragments thereof that specifically bind to an epitope on an alpha-synuclein peptide comprising the amino acid sequence of SEQ ID NO: 28. Also provided are isolated monoclonal antibodies or antigen-binding fragments thereof that specifically bind to an epitope on an alpha-synuclein peptide comprising the amino acid sequence of SEQ ID NO: 31.

In certain embodiments, the monoclonal antibody or antigen-binding fragment thereof reduces the level of alpha-synuclein.

In certain embodiments, the monoclonal antibody or antigen-binding fragment thereof prevents or reduces the level of alpha-synuclein aggregation.

Also provided are functional variants of the monoclonal antibodies or antigen-binding fragments thereof of the invention.

In certain embodiments, provided are immunoconjugates comprising the isolated monoclonal or antigen-binding fragment of the invention and at least one therapeutic and/or detectable agent.

Also provided are isolated nucleic acids encoding the monoclonal antibodies or antigen-binding fragment thereof of the invention disclosed herein.

Also provided are vectors comprising the isolated nucleic acids encoding the monoclonal antibodies or antigen-binding fragments thereof of the invention.

Also provided are host cells comprising the vectors comprising the isolated nucleic acids encoding the monoclonal antibodies or antigen-binding fragments thereof of the invention.

In certain embodiments, provided is a pharmaceutical composition comprising the isolated monoclonal antibody or antigen-binding fragment thereof of the invention and a pharmaceutically acceptable carrier.

Also provided are methods of preventing or reducing alpha-synuclein aggregation in a subject in need thereof, comprising administering to the subject the pharmaceutical compositions of the invention.

Also provided are methods of treating a disease characterized by Lewy bodies or alpha-synuclein aggregation in a subject in need thereof, comprising administering to the subject the pharmaceutical compositions of the invention. In certain embodiments, the disease characterized by Lewy bodies or alpha-synuclein aggregation is selected from any synucleinopathy. In other embodiments, the disease characterized by Lewy bodies or alpha-synuclein aggregation is selected from the group consisting of Alzheimer's disease, Parkinson's disease, dementia with Lewy bodies, multiple system atrophy, and lysosomal-storage diseases.

Also provided are methods of producing the monoclonal antibody or antigen-binding fragment thereof of the invention, comprising culturing a cell comprising a nucleic acid encoding the monoclonal antibody or antigen-binding fragment under conditions to produce the monoclonal antibody or antigen-binding fragment, and recovering the monoclonal antibody or antigen-binding fragment from the cell or culture.

Also provided are methods of producing a pharmaceutical composition comprising the monoclonal antibody or antigen-binding fragment thereof of the invention, comprising combining the monoclonal antibody or antigen-binding fragment with a pharmaceutically acceptable carrier to obtain the pharmaceutical composition.

Also provided are methods of determining a level of alpha-synuclein in a subject. The methods comprise (a) obtaining a sample from the subject; (b) contacting the sample with an isolated monoclonal antibody or antigen-binding fragment thereof of the invention; and (c) determining the level of alpha-synuclein in the subject. In certain embodiments, the sample is a tissue sample. The tissue sample can, for example, be a brain tissue sample. In certain embodiments, the sample is a blood sample.

In certain embodiments, provided are methods of diagnosing a disease characterized by Lewy bodies or alpha-synuclein aggregation. The methods comprise (a) obtaining a sample from the subject; (b) contacting the sample with an isolated monoclonal antibody or antigen-binding fragment thereof of the invention; and (c) detecting alpha-synuclein aggregates in the subject, wherein the detection of alpha-synuclein is diagnostic of the subject having a disease characterized by Lewy bodies or alpha-synuclein aggregates.

Also provided are kits comprising at least one isolated monoclonal antibody or antigen-binding fragment thereof of the invention.

### BRIEF DESCRIPTION OF THE DRAWINGS

The foregoing summary, as well as the following detailed description of preferred embodiments of the present application, will be better understood when read in conjunction with the appended drawings. It should be understood, however, that the application is not limited to the precise embodiments shown in the drawings.

FIGS. 1A-B show the sequence analysis of recovered anti-alpha-synuclein monoclonal antibodies. FIG. 1A shows the number of somatic mutations in amino acid (aa) and nucleotide (nt) sequences of the heavy chain (HC) and light chain (LC) variable regions of antibodies isolated from memory B cells from patients with Parkinson's disease (PD) and without Parkinson's disease (non-PD). Mutations and identification of the closest germline were determined using IgBlast databases. The horizontal lines indicate the mean. FIG. 1B shows phylogenetic analysis of recovered alpha-synuclein antibody heavy and light chain variable regions using the neighbor-joining algorithm (Jukes Cantor model) and illustrated as a circular tree.

FIG. 2 shows association (0-600 sec) and dissociation (600-1200 sec) profiles for a representative selection of recovered human anti-alpha-synuclein (hanti-Asyn) monoclonal antibodies to biotinylated full-length synuclein as determined by Octet biolayer interferometry. Data corresponding to individual hanti-Asyn variants are shown in the corresponding lines as highlighted in the figure legend.

FIGS. 3A-3C show the epitope mapping and specificity of hanti-Asyn-323.1, hanti-Asyn-336.1, and hanti-Asyn-338.1 as determined by Octet biolayer interferometry. Specificity was determined to peptide regions of alpha-synuclein covering amino acids 1-25 (syn1-25), 18-44 (syn18-44), 40-65 (syn40-65), 111-140 (syn111-140), 121-140 (syn121-140), and 111-140 with a phosphorylated serine at position 129 (syn11-140(pS129)). The association (0-600 sec) and dissociation (600-1200 sec) kinetics for the binding of hanti-Asyn-323.1 (FIG. 3A), hanti-ASyn-336.1 (FIG. 3B), and hanti-ASyn-338.1 (FIG. 3C) to different synuclein peptides are shown, and off-target binding of anti-synuclein mAbs against irrelevant tau peptides is shown in lower graph of each panel.

FIG. 4 shows the functional activity for a representative selection of hanti-Asyn monoclonal antibodies tested in an in vitro synuclein seeding assay. The assay measures the ability of each anti-synuclein mAb to inhibit the formation of synuclein aggregates in cells transiently expressing −V5 and −HA tagged full-length alpha-synuclein and treated with or without 10 μg/ml recombinant alpha-synuclein aggregates (seeds). Each antibody is tested at 500 μg/ml in the presence or absence of synuclein seeds and inhibitory activity is graphed as a percent of APC-positive particles. Each antibody was tested in quadruplicates across two independent experiments. Error bars indicate standard deviation (SD).

FIGS. 5A-5C show affinity binding of human anti-synuclein antibodies for full-length synuclein protein as determined by isothermal titration calorimetry (ITC). FIG. 5A shows binding affinity measurements for human anti-alpha-synuclein antibody 323.1 (hantiAsyn-323.1). FIG. 5B shows binding affinity measurements for human anti-alpha-synuclein antibody 336.1 (hantiAsyn-336.1). FIG. 5C shows binding affinity measurements for human anti-alpha-synuclein antibody 338.1 (hantiAsyn-338.1). The thermodynamic parameters and the equilibrium dissociation constants, Kd, were determined upon fitting the ITC data to a model assuming a single set of binding sites corresponding to an antibody:synuclein (1:2) binding model. Continuous lines represent the best fit of experimental data assuming a single set of binding sites. Experiments were performed in PBS. Equilibrium dissociation constants (K_{d}) are shown on the individual graphs.

FIG. 6 shows immunohistochemical detection of alpha-synuclein in Parkinson's Disease (PD) brain tissue. Immunohistochemistry was performed on the mesencephalon of a PD case. Panel A shows detection with control anti-synuclein mAb, LB509; panel B shows detection with hantiAsyn-336.1; panel C shows detection with hantiAsyn-338.1; and panel D shows detection with hantiAsyn-323.1. In panel B, the asterisk indicates a Lewy body (left from *, not stained with DAB). Scale bar represents 50 μm.

### DETAILED DESCRIPTION OF THE INVENTION

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the invention. Such discussion is not an admission that any or all of these matters form part of the prior art with respect to any inventions disclosed or claimed.

Unless defined otherwise, all technical and scientific terms used herein have the same meaning as commonly understood to one of ordinary skill in the art to which this invention pertains. Otherwise, certain terms used herein have the meanings as set forth in the specification.

It must be noted that as used herein and in the appended claims, the singular forms “a,” “an,” and “the” include plural reference unless the context clearly dictates otherwise.

Unless otherwise stated, any numerical values, such as a concentration or a concentration range described herein, are to be understood as being modified in all instances by the term “about.” Thus, a numerical value typically includes ±10% of the recited value. For example, a concentration of 1 mg/mL includes 0.9 mg/mL to 1.1 mg/mL. Likewise, a concentration range of 1% to 10% (w/v) includes 0.9% (w/v) to 11% (w/v). As used herein, the use of a numerical range expressly includes all possible subranges, all individual numerical values within that range, including integers within such ranges and fractions of the values unless the context clearly indicates otherwise.

Unless otherwise indicated, the term “at least” preceding a series of elements is to be understood to refer to every element in the series. Those skilled in the art will recognize or be able to ascertain using no more than routine experimentation, many equivalents to the specific embodiments of the invention described herein. Such equivalents are intended to be encompassed by the invention.

As used herein, the terms “comprises,” “comprising,” “includes,” “including,” “has,” “having,” “contains” or “containing,” or any other variation thereof, will be understood to imply the inclusion of a stated integer or group of integers but not the exclusion of any other integer or group of integers and are intended to be non-exclusive or open-ended. For example, a composition, a mixture, a process, a method, an article, or an apparatus that comprises a list of elements is not necessarily limited to only those elements but can include other elements not expressly listed or inherent to such composition, mixture, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

As used herein, the term “consists of,” or variations such as “consist of” or “consisting of,” as used throughout the specification and claims, indicate the inclusion of any recited integer or group of integers, but that no additional integer or group of integers can be added to the specified method, structure, or composition.

As used herein, the term “consists essentially of,” or variations such as “consist essentially of” or “consisting essentially of,” as used throughout the specification and claims, indicate the inclusion of any recited integer or group of integers, and the optional inclusion of any recited integer or group of integers that do not materially change the basic or novel properties of the specified method, structure or composition. See M.P.E.P. § 2111.03.

As used herein, “subject” means any animal, preferably a mammal, most preferably a human. The term “mammal” as used herein, encompasses any mammal. Examples of mammals include, but are not limited to, cows, horses, sheep, pigs, cats, dogs, mice, rats, rabbits, guinea pigs, monkeys, humans, etc., more preferably a human.

It should also be understood that the terms “about,” “approximately,” “generally,” “substantially” and like terms, used herein when referring to a dimension or characteristic of a component of the preferred invention, indicate that the described dimension/characteristic is not a strict boundary or parameter and does not exclude minor variations therefrom that are functionally the same or similar, as would be understood by one having ordinary skill in the art. At a minimum, such references that include a numerical parameter would include variations that, using mathematical and industrial principles accepted in the art (e.g., rounding, measurement or other systematic errors, manufacturing tolerances, etc.), would not vary the least significant digit.

The terms “identical” or percent “identity,” in the context of two or more nucleic acids or polypeptide sequences (e.g., anti-alpha-synuclein antibodies, alpha-synuclein polypeptides, and alpha-synuclein polynucleotides that encode them), refer to two or more sequences or subsequences that are the same or have a specified percentage of amino acid residues or nucleotides that are the same, when compared and aligned for maximum correspondence, as measured using one of the following sequence comparison algorithms or by visual inspection.

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally, Current Protocols in Molecular Biology, F. M. Ausubel et al., eds., Current Protocols, a joint venture between Greene Publishing Associates, Inc. and John Wiley & Sons, Inc., (1995 Supplement) (Ausubel)).

Examples of algorithms that are suitable for determining percent sequence identity and sequence similarity are the BLAST and BLAST 2.0 algorithms, which are described in Altschul et al. (1990) J. Mol. Biol. 215: 403-410 and Altschul et al. (1997) Nucleic Acids Res. 25: 3389-3402, respectively. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits act as seeds for initiating searches to find longer HSPs containing them. The word hits are then extended in both directions along each sequence for as far as the cumulative alignment score can be increased.

Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff, Proc. Natl. Acad. Sci. USA 89:10915 (1989)).

In addition to calculating percent sequence identity, the BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

A further indication that two nucleic acid sequences or polypeptides are substantially identical is that the polypeptide encoded by the first nucleic acid is immunologically cross reactive with the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules hybridize to each other under stringent conditions.

### Antibodies

The invention generally relates to isolated anti-alpha-synuclein antibodies, nucleic acids and expression vectors encoding the antibodies, recombinant cells containing the vectors, and compositions comprising the antibodies. Methods of making the antibodies, and methods of using the antibodies to diagnose and treat diseases characterized by Lewy bodies or alpha-synuclein aggregation. The antibodies of the invention possess one or more desirable functional properties, including but not limited to high-affinity binding to alpha-synuclein, the ability to reduce the level of alpha-synuclein, and ability to prevent or reduce alpha-synuclein aggregation.

In a general aspect, the invention relates to isolated monoclonal antibodies or antigen-binding fragments thereof that bind alpha-synuclein.

As used herein, the term “antibody” is used in a broad sense and includes immunoglobulin or antibody molecules including human, humanized, composite and chimeric antibodies and antibody fragments that are monoclonal or polyclonal. In general, antibodies are proteins or peptide chains that exhibit binding specificity to a specific antigen. Antibody structures are well known. Immunoglobulins can be assigned to five major classes (i.e., IgA, IgD, IgE, IgG and IgM), depending on the heavy chain constant domain amino acid sequence. IgA and IgG are further sub-classified as the isotypes IgA1, IgA2, IgG1, IgG2, IgG3 and IgG4. Accordingly, the antibodies of the invention can be of any of the five major classes or corresponding sub-classes. Preferably, the antibodies of the invention are IgG1, IgG2, IgG3 or IgG4. Antibody light chains of vertebrate species can be assigned to one of two clearly distinct types, namely kappa and lambda, based on the amino acid sequences of their constant domains. Accordingly, the antibodies of the invention can contain a kappa or lambda light chain constant domain. According to particular embodiments, the antibodies of the invention include heavy and/or light chain constant regions from human antibodies. In addition to the heavy and light constant domains, antibodies contain an antigen-binding region that is made up of a light chain variable region and a heavy chain variable region, each of which contains three domains (i.e., complementarity determining regions 1-3; CDR1, CDR2, and CDR3). The light chain variable region domains are alternatively referred to as LCDR1, LCDR2, and LCDR3, and the heavy chain variable region domains are alternatively referred to as HCDR1, HCDR2, and HCDR3.

As used herein, the term an “isolated antibody” refers to an antibody which is substantially free of other antibodies having different antigenic specificities (e.g., an isolated antibody that specifically binds to alpha-synuclein is substantially free of antibodies that do not bind to alpha-synuclein). In addition, an isolated antibody is substantially free of other cellular material and/or chemicals.

As used herein, the term “monoclonal antibody” refers to an antibody obtained from a population of substantially homogeneous antibodies, i.e., the individual antibodies comprising the population are identical except for possible naturally occurring mutations that may be present in minor amounts.

As used herein, the term “antigen-binding fragment” refers to an antibody fragment such as, for example, a diabody, a Fab, a Fab′, a F(ab′)2, an Fv fragment, a disulfide stabilized Fv fragment (dsFv), a (dsFv)₂, a bispecific dsFv (dsFv-dsFv′), a disulfide stabilized diabody (ds diabody), a single-chain antibody molecule (scFv), a single domain antibody (sdab) an scFv dimer (bivalent diabody), a multispecific antibody formed from a portion of an antibody comprising one or more CDRs, a camelized single domain antibody, a nanobody, a domain antibody, a bivalent domain antibody, or any other antibody fragment that binds to an antigen but does not comprise a complete antibody structure. An antigen-binding fragment is capable of binding to the same antigen to which the parent antibody or a parent antibody fragment binds. According to particular embodiments, the antigen-binding fragment comprises a light chain variable region, a light chain constant region, and an Fd segment of the heavy chain. According to other particular embodiments, the antigen-binding fragment comprises Fab and F(ab′).

As used herein, the term “human antibody” refers to an antibody produced by a human or an antibody having an amino acid sequence corresponding to an antibody produced by a human made using any technique known in the art. This definition of a human antibody includes intact or full-length antibodies, fragments thereof, and/or antibodies comprising at least one human heavy and/or light chain polypeptide. Compared to artificially generated human-like antibodies such as single chain antibody fragments (scFvs) from a phage displayed antibody library or xenogeneic mice, the human antibody of the present invention is characterized by (i) the antigen-binding region being obtained using the human immune response rather than that of animal surrogates, i.e. the antigen binding region has been generated in response to natural alpha-synuclein in its relevant conformation in the human body, and/or (ii) having protected the individual or is at least significant for the presence of alpha-synuclein.

For example, the paring of heavy and light chains of human-like antibodies such as synthetic and semi-synthetic antibodies typically isolated from phage display do not necessarily reflect the original paring as it occurred in the original human B cell. Accordingly, Fab and scFv fragments obtained from recombinant expression libraries as commonly used in the prior art can be considered as being artificial with all possible associated effects on immunogenicity and stability. In contrast, the present invention provides antigen-binding regions of affinity-matured anti-alpha-synuclein antibodies from selected human subjects, which, in certain embodiments, are recombinantly expressed as chimeras with a common IgG1 constant region.

As used herein, the terms “alpha-synuclein” or “α-synuclein” are used interchangeably and refer to the human alpha-synuclein protein, which is a member of a protein family of synucleins. Alpha-synuclein is a highly soluble natively unfolded protein expressed throughout the central nervous system. Under pathological conditions, alpha-synuclein forms insoluble fibers, or protofibrils, which aggregate and form the main structural component of Lewy bodies (Spillantini et al. 1997; Spillantini et al. 1998; Baba et al. 1998). The spreading of alpha-synuclein aggregates has been correlated with disease progression (Braak et al. 2003). The protein is composed of three distinct regions: (1) an amino terminus (residues 1-60), containing apolipoprotein lipid-binding motifs, which are predicted to form amphiphilic helices conferring the propensity to form α-helical structures on membrane binding, (2) a central hydrophobic region (61-95), so-called NAC (non-Aβ component), which confers the β-sheet potential, and (3) a carboxyl terminus that is highly negatively charged, and is prone to be unstructured. The SNCA gene encodes for the 140 amino acid alpha-synuclein protein. Point mutations (A30P, E46K, H50Q, G51D, A53E and A53T) of alpha-synuclein protein and increased dosage of SNCA, the gene encoding alpha-synuclein, are associated with the familial form of PD (Lashuel et al., 2013; Wong and Krainc, 2017). Moreover, a genome-wide association study (GWAS) identified SNCA (accession number NM_000345) as one of the most important genetic risk factors for idiopathic PD (Goedert et al., 2013).

As used herein, an antibody that “specifically binds to alpha-synuclein” refers to an antibody that binds to an alpha-synuclein, preferably a human alpha-synuclein, with a KD of 1×10⁻⁵ M or less, preferably 5×10⁻⁶ M or less, more preferably 1×10⁻⁷ M or less, preferably 1×10⁻⁸ M or less, more preferably 5×10⁻⁹ M or less, 1×10⁻⁹ M or less, 5×10⁻¹⁰ M or less, or 1×10⁻¹⁰ M or less. The term “KD” refers to the dissociation constant, which is obtained from the ratio of Kd to Ka (i.e., Kd/Ka) and is expressed as a molar concentration (M). KD values for antibodies can be determined using methods in the art in view of the present disclosure. For example, the KD of an antibody can be determined by using surface plasmon resonance, such as by using a biosensor system, e.g., a Biacore® system, or by using bio-layer interferometry technology, such as an Octet RED96 system.

The smaller the value of the KD of an antibody, the higher affinity that the antibody binds to a target antigen. As used herein, the term “affinity” refers to a measure of the strength of the binding of an individual epitope or partial epitope with the CDRs of a binding molecule, e.g., an immunoglobulin molecule; see, e.g., Harlow et al., Antibodies: A Laboratory Manual, Cold Spring Harbor Laboratory Press, 2nd ed. (1988) at pages 27-28. General techniques for measuring the affinity of an antibody for an antigen include enzyme-linked immunosorbent assays (ELISA), radioimmunoassays (RIA), isothermal titration calorimetry (ITC), and surface plasmon resonance.

The term “epitope” as used herein means that part of the antigen that is contacted by the CDR loops of the antibody. A “structural epitope” comprises about 15-22 contact residues on the antigen surface and involves many amino acid residues that make contact with a large group of residues on the CDRs collectively referred to as the paratope of antibody. Direct contact between epitope and paratope residues is established through electrostatic forces such as hydrogen bonds, salt bridges, van der Waals forces of hydrophobic surfaces and shape complementarity The interface has also bound water molecules or other co-factors that contribute to the specificity and affinity of antigen-antibody interactions The binding energy of an antigen-antibody complex is primarily mediated by a small subset of contact residues in the epitope-paratope interface. These “energetic residues” are often located in the center of the epitope-paratope interface and make up the functional epitope. Contact residues in the periphery of the interface make generally minor contributions to the binding energy; their replacements have frequently little effect on the binding with antigen. Thus, the binding or functional activity of an epitope involves a small subset of energetic residues centrally located in the structural epitope and contacted by the specificity-determining CDRs. The assignment of a functional epitope on an antigenic protein can be made using several methods including Alanine scanning mutagenesis or by solving the crystal structure of the antigen with the antibody.

An epitope can be linear in nature or can be a discontinuous epitope, e.g., a conformational epitope, which is formed by a spatial relationship between non-contiguous amino acids of an antigen rather than a linear series of amino acids. A conformational epitope includes epitopes resulting from the folding of an antigen, where amino acids from differing portions of the linear sequence of the antigen come in close proximity in the three-dimensional space. For discontinuous epitopes, it can be possible to obtain binding of one or more linear peptides with decreased affinity to a so-called partial epitope, e. g. dispersed at different regions of the protein sequence (M. S. Cragg (2011), Blood 118 (2):219-20).

According to a particular aspect, the invention relates to an isolated monoclonal antibody or antigen-binding fragment thereof comprising a heavy chain complementarity determining region 1 (HCDR1), a HCDR2, a HCDR3, a light chain complementarity determining region 1 (LCDR1), a LCDR2, and a LCDR3, having the polypeptide sequences of:
(a) SEQ ID NOs: 7, 8, 9, 16, 17, and 18, respectively;
(b) SEQ ID NOs: 10, 11, 12, 19, 20, and 21, respectively; or
(c) SEQ ID NOs: 13, 14, 15, 22, 23, and 24, respectively;
   wherein the antibody or antigen-binding fragment thereof specifically binds alpha-synuclein, preferably human alpha-synuclein.

According to another particular aspect, the invention relates to an isolated monoclonal antibody or antigen-binding fragment thereof of the invention, comprising:
(a) a heavy chain variable region having the polypeptide sequence of SEQ ID NO:1, and a light chain variable region having the polypeptide sequence of SEQ ID NO:2;
(b) a heavy chain variable region having the polypeptide sequence of SEQ ID NO:3, and a light chain variable region having the polypeptide sequence of SEQ ID NO:4; or
(c) a heavy chain variable region having the polypeptide sequence of SEQ ID NO:5, and a light chain variable region having the polypeptide sequence of SEQ ID NO:6.

In one embodiment, the invention relates to an isolated monoclonal antibody or antigen-binding fragment thereof, comprising HCDR1, HCDR2, HCDR3, LCDR1, LCDR2 and LCDR3, having the polypeptide sequences of SEQ ID NOs: 7, 8, 9, 16, 17, and 18, respectively. In another embodiment, the isolated monoclonal antibody or antigen-binding fragment thereof comprises a heavy chain variable region having a polypeptide sequence at least 85%, preferably 90%, more preferably 95% or more, such as 95%, 96%, 97%, 98%, or 99% identical to SEQ ID NO:1, and a light chain variable region having a polypeptide sequence at least 85%, preferably 90%, more preferably 95% or more, such as 95%, 96%, 97%, 98%, or 99% identical to SEQ ID NO:2. Preferably, the isolated monoclonal antibody or antigen-binding fragment thereof comprises a heavy chain variable region having the polypeptide sequence of SEQ ID NO:1; and a light chain variable region having the polypeptide sequence of SEQ ID NO:2.

In one embodiment, the invention relates to an isolated monoclonal antibody or antigen-binding fragment thereof, comprising HCDR1, HCDR2, HCDR3, LCDR1, LCDR2 and LCDR3, having the polypeptide sequences of SEQ ID NOs: 10, 11, 12, 19, 20, and 21, respectively. In another embodiment, the isolated monoclonal antibody or antigen-binding fragment thereof comprises a heavy chain variable region having a polypeptide sequence at least 85%, preferably 90%, more preferably 95% or more, such as 95%, 96%, 97%, 98%, or 99% identical to SEQ ID NO:3, and a light chain variable region having a polypeptide sequence at least 85%, preferably 90%, more preferably 95% or more, such as 95%, 96%, 97%, 98%, or 99% identical to SEQ ID NO:4. Preferably, the isolated monoclonal antibody or antigen-binding fragment thereof comprises a heavy chain variable region having the polypeptide sequence of SEQ ID NO:3; and a light chain variable region having the polypeptide sequence of SEQ ID NO:4.

In one embodiment, the invention relates to an isolated monoclonal antibody or antigen-binding fragment thereof, comprising HCDR1, HCDR2, HCDR3, LCDR1, LCDR2 and LCDR3, having the polypeptide sequences of SEQ ID NOs: 13, 14, 15, 22, 23, and 24, respectively. In another embodiment, the isolated monoclonal antibody or antigen-binding fragment thereof comprises a heavy chain variable region having a polypeptide sequence at least 85%, preferably 90%, more preferably 95% or more, such as 95%, 96%, 97%, 98%, or 99% identical to SEQ ID NO:5, and a light chain variable region having a polypeptide sequence at least 85%, preferably 90%, more preferably 95% or more, such as 95%, 96%, 97%, 98%, or 99% identical to SEQ ID NO:6. Preferably, the isolated monoclonal antibody or antigen-binding fragment thereof comprises a heavy chain variable region having the polypeptide sequence of SEQ ID NO:5; and a light chain variable region having the polypeptide sequence of SEQ ID NO:6.

According to another particular aspect, the invention relates to an isolated monoclonal antibody or antigen-binding fragment thereof that specifically binds to an epitope on an alpha-synuclein peptide comprising the amino acid sequence of SEQ ID NO: 28. According to another particular aspect, the invention relates to an isolated monoclonal antibodies or antigen-binding fragments thereof that specifically binds to an epitope on an alpha-synuclein peptide comprising the amino acid sequence of SEQ ID NO: 31.

In a general aspect, the invention relates to functional variants of the isolated monoclonal antibody or antigen-binding fragment thereof. The term “functional variant,” as used herein, refers to an antibody that comprises a nucleotide and/or amino acid sequence that is altered by one or more nucleotides and/or amino acids compared to the nucleotide and/or amino acid sequences of a reference antibody and that is capable of competing for specific binding to the binding partner, i.e. alpha-synuclein, with the reference antibody. In other words, the modifications in the amino acid and/or nucleotide sequence of the reference antibody do not significantly affect or alter the binding characteristics of the antibody encoded by the nucleotide sequence or containing the amino acid sequence, i.e. the antibody is still able to specifically recognize and bind its target. The functional variant may have conservative sequence modifications including nucleotide and amino acid substitutions, additions and deletions. Examples of functional variants include derisking a free cysteine or amino acid with potential post-translational modification in the hypervariable region, as well as Fc engineering to increase/decrease serum half-life and/or the binding affinity of IgG antibodies to FcRn. A functional variant can also include the generation of the antibody as a human chimeric IgG2, IgG3 or IgG4 isotype, or as a chimeric isotype of a different species. These modifications can be introduced by standard techniques known in the art, such as PCR, site-directed mutagenesis, and random PCR-mediated mutagenesis, and can comprise natural as well as non-natural nucleotides and amino acids.

In another general aspect, the invention provides immunoconjugates, or antibody-drug conjugates (ADC), comprising an antibody conjugated to a cytotoxic agent such as a chemotherapeutic agent, a drug, a growth inhibitory agent, a toxin (e.g., an enzymatically active toxin of bacterial, fungal, plant, or animal origin, or fragments thereof), or a radioactive isotope (i.e., a radioconjugate). According to a particular aspect, an immunoconjugate comprises any of the above antibodies covalently attached to at least one therapeutic and/or detectable agents.

According to another particular aspect of the invention, the invention relates to an isolated monoclonal antibody or antigen-binding fragment thereof, wherein the monoclonal antibody or antigen-binding fragment thereof reduces the level of alpha-synuclein.

According to another particular aspect of the invention, the invention relates to an isolated monoclonal antibody or antigen-binding fragment thereof, wherein the monoclonal antibody or antigen-binding fragment thereof prevents or reduces the level of alpha-synuclein aggregation.

In another general aspect, the invention relates to an isolated nucleic acid encoding a monoclonal antibody or antigen-binding fragment thereof of the invention. It will be appreciated by those skilled in the art that the coding sequence of a protein can be changed (e.g., replaced, deleted, inserted, etc.) without changing the amino acid sequence of the protein. Accordingly, it will be understood by those skilled in the art that nucleic acid sequences encoding monoclonal antibodies or antigen-binding fragments thereof of the invention can be altered without changing the amino acid sequences of the proteins.

In another general aspect, the invention relates to a vector comprising an isolated nucleic acid encoding a monoclonal antibody or antigen-binding fragment thereof of the invention. Any vector known to those skilled in the art in view of the present disclosure can be used, such as a plasmid, a cosmid, a phage vector or a viral vector. In some embodiments, the vector is a recombinant expression vector such as a plasmid. The vector can include any element to establish a conventional function of an expression vector, for example, a promoter, ribosome binding element, terminator, enhancer, selection marker, and origin of replication. The promoter can be a constitutive, inducible or repressible promoter. A number of expression vectors capable of delivering nucleic acids to a cell are known in the art and can be used herein for production of an antibody or antigen-binding fragment thereof in the cell. Conventional cloning techniques or artificial gene synthesis can be used to generate a recombinant expression vector according to embodiments of the invention.

In another general aspect, the invention relates to a host cell comprising an isolated nucleic acid encoding a monoclonal antibody or antigen-binding fragment thereof of the invention. Any host cell known to those skilled in the art in view of the present disclosure can be used for recombinant expression of antibodies or antigen-binding fragments thereof of the invention. In some embodiments, the host cells are E. coli DH5α or BL21 cells (for expression of, e.g., an scFv or Fab antibody), HEK293 cells (for expression of, e.g., a full-length IgG antibody). According to particular embodiments, the recombinant expression vector is transformed into host cells by conventional methods such as chemical transfection, heat shock, or electroporation, where it is stably integrated into the host cell genome such that the recombinant nucleic acid is effectively expressed.

In another general aspect, the invention relates to a method of producing a monoclonal antibody or antigen-binding fragment thereof of the invention, comprising culturing a cell comprising a nucleic acid encoding the monoclonal antibody or antigen-binding fragment thereof under conditions to produce a monoclonal antibody or antigen-binding fragment thereof of the invention, and recovering the antibody or antigen-binding fragment thereof from the cell or cell culture (e.g., from the supernatant). Expressed antibodies or antigen-binding fragments thereof can be harvested from the cells and purified according to conventional techniques known in the art and as described herein.

### Pharmaceutical Compositions

In another general aspect, the invention relates to a pharmaceutical composition, comprising an isolated monoclonal antibody or antigen-binding fragment thereof of the invention and a pharmaceutically acceptable carrier. The term “pharmaceutical composition” as used herein means a product comprising an antibody of the invention together with a pharmaceutically acceptable carrier. Antibodies of the invention and compositions comprising them are also useful in the manufacture of a medicament for therapeutic applications mentioned herein.

As used herein, the term “carrier” refers to any excipient, diluent, filler, salt, buffer, stabilizer, solubilizer, oil, lipid, lipid containing vesicle, microsphere, liposomal encapsulation, or other material well known in the art for use in pharmaceutical formulations. It will be understood that the characteristics of the carrier, excipient or diluent will depend on the route of administration for a particular application. As used herein, the term “pharmaceutically acceptable carrier” refers to a non-toxic material that does not interfere with the effectiveness of a composition according to the invention or the biological activity of a composition according to the invention. According to particular embodiments, in view of the present disclosure, any pharmaceutically acceptable carrier suitable for use in an antibody pharmaceutical composition can be used in the invention.

The formulation of pharmaceutically active ingredients with pharmaceutically acceptable carriers is known in the art, e.g., Remington: The Science and Practice of Pharmacy (e.g. 21st edition (2005), and any later editions). Non-limiting examples of additional ingredients include: buffers, diluents, solvents, tonicity regulating agents, preservatives, stabilizers, and chelating agents. One or more pharmaceutically acceptable carrier may be used in formulating the pharmaceutical compositions of the invention.

In one embodiment of the invention, the pharmaceutical composition is a liquid formulation. A preferred example of a liquid formulation is an aqueous formulation, i.e., a formulation comprising water. The liquid formulation may comprise a solution, a suspension, an emulsion, a microemulsion, a gel, and the like. An aqueous formulation typically comprises at least 50% w/w water, or at least 60%, 70%. 75%. 80%. 85%. 90%, or at least 95% w/w of water.

In one embodiment, the pharmaceutical composition may be formulated as an injectable which can be injected, for example, via an injection device (e.g., a syringe or an infusion pump). The injection may be delivered subcutaneously, intramuscularly, intraperitoneally, intravitreally, or intravenously, for example.

In another embodiment, the pharmaceutical composition is a solid formulation, e.g., a freeze-dried or spray-dried composition, which may be used as is, or whereto the physician or the patient adds solvents, and/or diluents prior to use. Solid dosage forms may include tablets, such as compressed tablets, and/or coated tablets, and capsules (e.g., hard or soft gelatin capsules). The pharmaceutical composition may also be in the form of sachets, dragees, powders, granules, lozenges, or powders for reconstitution, for example.

In other embodiments, the pharmaceutical composition may be delivered intranasally, intrabuccally, or sublingually.

The pH in an aqueous formulation can be between pH 3 and pH 10. In one embodiment of the invention, the pH of the formulation is from about 7.0 to about 9.5. In another embodiment of the invention, the pH of the formulation is from about 3.0 to about 7.0.

In another embodiment of the invention, the pharmaceutical composition comprises a buffer. Non-limiting examples of buffers include: arginine, aspartic acid, bicine, citrate, disodium hydrogen phosphate, fumaric acid, glycine, glycylglycine, histidine, lysine, maleic acid, malic acid, sodium acetate, sodium carbonate, sodium dihydrogen phosphate, sodium phosphate, succinate, tartaric acid, tricine, and tris(hydroxymethyl)-aminomethane, and mixtures thereof. The buffer may be present individually or in the aggregate, in a concentration from about 0.01 mg/ml to about 50 mg/ml, for example from about 0.1 mg/ml to about 20 mg/ml. Pharmaceutical compositions comprising each one of these specific buffers constitute alternative embodiments of the invention.

In another embodiment of the invention, the pharmaceutical composition comprises a preservative. Non-limiting examples of preservatives include: benzethonium chloride, benzoic acid, benzyl alcohol, bronopol, butyl 4-hydroxybenzoate, chlorobutanol, chlorocresol, chlorohexidine, chlorphenesin, o-cresol, m-cresol, p-cresol, ethyl 4-hydroxybenzoate, imidurea, methyl 4-hydroxybenzoate, phenol, 2-phenoxyethanol, 2-phenylethanol, propyl 4-hydroxybenzoate, sodium dehydroacetate, thiomerosal, and mixtures thereof. The preservative may be present individually or in the aggregate, in a concentration from about 0.01 mg/ml to about 50 mg/ml, for example from about 0.1 mg/ml to about 20 mg/ml. Pharmaceutical compositions comprising each one of these specific preservatives constitute alternative embodiments of the invention.

In another embodiment of the invention, the pharmaceutical composition comprises an isotonic agent. Non-limiting examples of the embodiment include a salt (such as sodium chloride), an amino acid (such as glycine, histidine, arginine, lysine, isoleucine, aspartic acid, tryptophan, and threonine), an alditol (such as glycerol, 1,2-propanediol propyleneglycol), 1,3-propanediol, and 1,3-butanediol), polyethyleneglycol (e.g. PEG400), and mixtures thereof. Another example of an isotonic agent includes a sugar. Non-limiting examples of sugars may be mono-, di-, or polysaccharides, or water-soluble glucans, including for example fructose, glucose, mannose, sorbose, xylose, maltose, lactose, sucrose, trehalose, dextran, pullulan, dextrin, cyclodextrin, alpha and beta-HPCD, soluble starch, hydroxyethyl starch, and sodium carboxymethylcellulose. Another example of an isotonic agent is a sugar alcohol, wherein the term “sugar alcohol” is defined as a C(4-8) hydrocarbon having at least one —OH group. Non-limiting examples of sugar alcohols include mannitol, sorbitol, inositol, galactitol, dulcitol, xylitol, and arabitol. Pharmaceutical compositions comprising each isotonic agent listed in this paragraph constitute alternative embodiments of the invention. The isotonic agent may be present individually or in the aggregate, in a concentration from about 0.01 mg/ml to about 50 mg/ml, for example from about 0.1 mg/ml to about 20 mg/ml. Pharmaceutical compositions comprising each one of these specific isotonic agents constitute alternative embodiments of the invention.

In another embodiment of the invention, the pharmaceutical composition comprises a chelating agent. Non-limiting examples of chelating agents include citric acid, aspartic acid, salts of ethylenediaminetetraacetic acid (EDTA), and mixtures thereof. The chelating agent may be present individually or in the aggregate, in a concentration from about 0.01 mg/ml to about 50 mg/ml, for example from about 0.1 mg/ml to about 20 mg/ml. Pharmaceutical compositions comprising each one of these specific chelating agents constitute alternative embodiments of the invention.

In another embodiment of the invention, the pharmaceutical composition comprises a stabilizer. Non-limiting examples of stabilizers include one or more aggregation inhibitors, one or more oxidation inhibitors, one or more surfactants, and/or one or more protease inhibitors.

In another embodiment of the invention, the pharmaceutical composition comprises a stabilizer, wherein said stabilizer is carboxy-/hydroxycellulose and derivatives thereof (such as HPC, HPC-SL, HPC-L and HPMC), cyclodextrins, 2-methylthioethanol, polyethylene glycol (such as PEG 3350), polyvinyl alcohol (PVA), polyvinyl pyrrolidone, salts (such as sodium chloride), sulphur-containing substances such as monothioglycerol), or thioglycolic acid. The stabilizer may be present individually or in the aggregate, in a concentration from about 0.01 mg/ml to about 50 mg/ml, for example from about 0.1 mg/ml to about 20 mg/ml. Pharmaceutical compositions comprising each one of these specific stabilizers constitute alternative embodiments of the invention.

In further embodiments of the invention, the pharmaceutical composition comprises one or more surfactants, preferably a surfactant, at least one surfactant, or two different surfactants. The term “surfactant” refers to any molecules or ions that are comprised of a water-soluble (hydrophilic) part, and a fat-soluble (lipophilic) part. The surfactant may, for example, be selected from the group consisting of anionic surfactants, cationic surfactants, nonionic surfactants, and/or zwitterionic surfactants. The surfactant may be present individually or in the aggregate, in a concentration from about 0.1 mg/ml to about 20 mg/ml. Pharmaceutical compositions comprising each one of these specific surfactants constitute alternative embodiments of the invention.

In a further embodiment of the invention, the pharmaceutical composition comprises one or more protease inhibitors, such as, e.g., EDTA, and/or benzamidine hydrochloric acid (HCl). The protease inhibitor may be present individually or in the aggregate, in a concentration from about 0.1 mg/ml to about 20 mg/ml. Pharmaceutical compositions comprising each one of these specific protease inhibitors constitute alternative embodiments of the invention.

In another general aspect, the invention relates to a method of producing a pharmaceutical composition comprising a monoclonal antibody or antigen-binding fragment thereof of the invention, comprising combining a monoclonal antibody or antigen-binding fragment thereof with a pharmaceutically acceptable carrier to obtain the pharmaceutical composition.

### Methods of Use

In another general aspect, the invention relates to a method of preventing or reducing alpha-synuclein aggregation in a subject in need thereof, comprising administering to the subject an isolated monoclonal antibody or antigen binding fragment thereof that specifically binds alpha-synuclein or a pharmaceutical composition of the invention.

The functional activity of antibodies and antigen-binding fragments thereof that bind alpha-synuclein can be characterized by methods known in the art and as described herein. Methods for characterizing antibodies and antigen-binding fragments thereof that bind alpha-synuclein include, but are not limited to, affinity and specificity assays including Biacore, ELISA, and OctetRed analysis; surface plasmon resonance (SPR) assays. The functional activity of an anti-alpha-synuclein mAb can also be assessed in an intracellular alpha-synuclein aggregation assay, wherein cells are incubated with misfolded recombinant alpha-synuclein seeds and anti-alpha-synuclein antibody to determine whether the antibody can block alpha-synuclein aggregate uptake and intracellular synuclein aggregation. According to particular embodiments, the methods for characterizing antibodies and antigen-binding fragments thereof that bind alpha-synuclein include those described below.

The antibodies of the invention are suitable both as therapeutic and prophylactic agents for treating or preventing disease that involve pathological aggregation of alpha-synuclein. In another general aspect, the invention relates to a method of treating or preventing a disease characterized by Lewy bodies or alpha-synuclein aggregation in a subject in need thereof, comprising administering to the subject an isolated monoclonal antibody or antigen binding fragment thereof that specifically binds alpha-synuclein or a pharmaceutical composition of the invention. Lewy bodies are cytoplasmic inclusions containing alpha-synuclein fibrils aggregated to form an insoluble mass located inside neural cells. Diseases characterized by the presence of Lewy bodies or alpha-synuclein aggregates are known collectively as Lewy body diseases or synucleinopathies. As used herein, a “synucleinopathy” encompasses any neurodegenerative disease that involves the pathological aggregation of alpha-synuclein. In particular embodiments, the disease can, for example, be selected from but not limited to, Alzheimer's disease, Parkinson's disease, dementia with Lewy bodies, multiple system atrophy, pure autonomic failure, lysosomal-storage diseases, and other synuclein-related pathologies.

According to embodiments of the invention, the pharmaceutical composition comprises a therapeutically effective amount of an anti-alpha-synuclein antibody or antigen-binding fragment thereof. As used herein, the term “therapeutically effective amount” refers to an amount of an active ingredient or component that elicits the desired biological or medicinal response in a subject. A therapeutically effective amount can be determined empirically and in a routine manner, in relation to the stated purpose.

According to particular embodiments, a therapeutically effective amount refers to the amount of therapy which is sufficient to achieve one, two, three, four, or more of the following effects: (i) reduce or ameliorate the severity of the disease, disorder or condition to be treated or a symptom associated therewith; (ii) reduce the duration of the disease, disorder or condition to be treated, or a symptom associated therewith; (iii) prevent the progression of the disease, disorder or condition to be treated, or a symptom associated therewith; (iv) cause regression of the disease, disorder or condition to be treated, or a symptom associated therewith; (v) prevent the development or onset of the disease, disorder or condition to be treated, or a symptom associated therewith; (vi) prevent the recurrence of the disease, disorder or condition to be treated, or a symptom associated therewith; (vii) reduce hospitalization of a subject having the disease, disorder or condition to be treated, or a symptom associated therewith; (viii) reduce hospitalization length of a subject having the disease, disorder or condition to be treated, or a symptom associated therewith; (ix) increase the survival of a subject with the disease, disorder or condition to be treated, or a symptom associated therewith; (xi) inhibit or reduce the disease, disorder or condition to be treated, or a symptom associated therewith in a subject; and/or (xii) enhance or improve the prophylactic or therapeutic effect(s) of another therapy.

The therapeutically effective amount or dosage can vary according to various factors, such as the disease, disorder or condition to be treated, the means of administration, the target site, the physiological state of the subject (including, e.g., age, body weight, health), whether the subject is a human or an animal, other medications administered, and whether the treatment is prophylactic or therapeutic. Treatment dosages are optimally titrated to optimize safety and efficacy.

According to particular embodiments, the compositions described herein are formulated to be suitable for the intended route of administration to a subject. For example, the compositions described herein can be formulated to be suitable for intravenous, subcutaneous, or intramuscular administration.

As used herein, the terms “treat,” “treating,” and “treatment” are all intended to refer to an amelioration or reversal of at least one measurable physical parameter related to a disease characterized by Lewy bodies or alpha-synuclein aggregation, which is not necessarily discernible in the subject, but can be discernible in the subject. The terms “treat,” “treating,” and “treatment,” can also refer to causing regression, preventing the progression, or at least slowing down the progression of the disease, disorder, or condition. In a particular embodiment, “treat.” “treating,” and “treatment” refer to an alleviation, prevention of the development or onset, or reduction in the duration of one or more symptoms associated with the disease, disorder, or condition. In a particular embodiment, “treat,” “treating,” and “treatment” refer to reducing the risk of, lessening the severity of, or delaying the outset a disease, disorder, or condition. In a particular embodiment, “treat,” “treating,” and “treatment” refer to an increase in the survival of a subject having the disease, disorder, or condition. In a particular embodiment, “treat,” “treating,” and “treatment” refer to elimination of the disease, disorder, or condition in the subject.

In another general aspect, the invention relates to a method of determining a level of alpha-synuclein in a subject. The methods comprise (a) obtaining a sample from the subject; (b) contacting the sample with an isolated monoclonal antibody or antigen-binding fragment thereof of the invention; and (c) determining the level of alpha-synuclein in the subject.

As used herein, “sample” refers to a biological sample isolated from a subject and can include, but is not limited to, whole blood, serum, plasma, blood cells, endothelial cells, tissue biopsies (e.g., brain tissue), lymphatic fluid, ascites fluid, interstitial fluid, bone marrow, cerebrospinal fluid, saliva, mucous, sputum, sweat, urine, or any other secretion, excretion, or other bodily fluids. A “blood sample” refers to whole blood or any fraction thereof, including blood cells, serum, and plasma.

In certain embodiments, the level of alpha-synuclein in the subject can be determined utilizing assays selected from, but not limited to, a Western blot assay, an ELISA assay, a FACS assay, and/or a radioimmunoassay (RIA). Relative protein levels can be determined by utilizing Western blot analysis, FACS assay, and immunohistochemistry (IHC), in vivo imaging, and absolute protein levels can be determined by utilizing an ELISA assay. When determining the relative levels of alpha-synuclein, the levels of alpha-synuclein can be determined between at least two samples, e.g., between samples from the same subject at different time points, between samples from different tissues in the same subject, and/or between samples from different subjects. Alternatively, when determining absolute levels of alpha-synuclein, such as by an ELISA assay, the absolute level of alpha-synuclein in the sample can be determined by creating a standard for the ELISA assay prior to testing the sample. A person skilled in the art would understand which analytical techniques to utilize to determine the level of alpha-synuclein in a sample from the subject utilizing the antibodies or antigen-binding fragments thereof of the invention.

Utilizing methods of determining a level of alpha-synuclein in a sample from a subject can lead to the diagnosis of abnormal (elevated, reduced, or insufficient) alpha-synuclein levels in a disease and making appropriate therapeutic decisions. Such a disease can be selected from, Alzheimer's disease, Parkinson's disease, dementia with Lewy bodies, multiple system atrophy, and lysosomal-storage diseases. Additionally, by monitoring the levels of alpha-synuclein in a subject, the risk of developing a disease as indicated above can be determined based on the knowledge of the level of alpha-synuclein in a particular disease and/or during the progression of the particular disease.

Diagnostic antibodies or similar reagents can be administered by intravenous injection into the body of the patient, or directly into the brain by any suitable route that delivers the agent to the host as exemplified above. The dosage of antibody should be within the same ranges as for treatment methods. Typically, the antibody is labeled, although in some methods, the primary antibody with affinity for alpha-synuclein is unlabeled and a secondary labeling agent is used to bind to the primary antibody. The choice of label depends on the means of detection. For example, a fluorescent label is suitable for optical detection. Use of paramagnetic labels is suitable for tomographic detection without surgical intervention. Radioactive labels can also be detected using PET or SPECT.

Diagnosis is performed by comparing the number, size, and/or intensity of labeled alpha-synuclein, alpha-synuclein aggregates, and/or Lewy bodies in a sample from the subject or in the subject, to corresponding baseline values. The baseline values can represent the mean levels in a population of non-diseased individuals. Baseline values can also represent previous levels determined in the same subject.

The diagnostic methods described above can also be used to monitor a subject's response to therapy by detecting the presence of alpha-synuclein in a subject before, during or after the treatment. A change in values relative to baseline signals a response to treatment. Values can also change temporarily in biological fluids as pathological alpha-synuclein is being cleared from the brain.

The present invention is further directed to a kit for performing the above described diagnostic and monitoring methods. Typically, such kits contain a diagnostic reagent such as the antibodies of the invention, and optionally a detectable label. The diagnostic antibody itself may contain the detectable label (e.g., fluorescent molecule, biotin, etc.) which is directly detectable or detectable via a secondary reaction (e.g., reaction with streptavidin). Alternatively, a second reagent containing the detectable label may be utilized, where the second reagent has binding specificity for the primary antibody. In a diagnostic kit suitable for measuring alpha-synuclein in a biological sample, the antibodies of the kit may be supplied pre-bound to a solid phase, such as to the wells of a microtiter dish.

### EMBODIMENTS

The invention provides also the following non-limiting embodiments.

Embodiment 1 is an isolated monoclonal antibody or antigen-binding fragment thereof comprising a heavy chain complementarity determining region 1 (HCDR1), HCDR2, HCDR3, a light chain complementarity determining region 1 (LCDR1), LCDR2, and LCDR3, having the polypeptide sequences of:
(a) SEQ ID NOs: 7, 8, 9, 16, 17, and 18, respectively;
(b) SEQ ID NOs: 10, 11, 12, 19, 20, and 21, respectively; or
(c) SEQ ID NOs: 13, 14, 15, 22, 23, and 24, respectively;
   wherein the antibody or antigen-binding fragment thereof specifically binds alpha-synuclein, preferably human alpha-synuclein.

Embodiment 2 is a monoclonal antibody or antigen-binding fragment thereof of embodiment 1, comprising a heavy chain variable region having a polypeptide sequence at least 95% identical to SEQ ID NO: 1, 3, or 5, or a light chain variable region having a polypeptide sequence at least 95% identical to SEQ ID NO: 2, 4, or 6.

Embodiment 3 is the isolated monoclonal antibody or antigen-binding fragment of embodiment 1 or 2, comprising
(a) a heavy chain variable region having the polypeptide sequence of SEQ ID NO:1, and a light chain variable region having the polypeptide sequence of SEQ ID NO:2;
(b) a heavy chain variable region having the polypeptide sequence of SEQ ID NO:3, and a light chain variable region having the polypeptide sequence of SEQ ID NO:4; or
(c) a heavy chain variable region having the polypeptide sequence of SEQ ID NO:5, and a light chain variable region having the polypeptide sequence of SEQ ID NO:6.

Embodiment 4 is an isolated monoclonal antibody or antigen-binding fragment thereof of any one of embodiments 1-3 that specifically binds to an epitope on an alpha-synuclein peptide comprising the amino acid sequence of SEQ ID NO: 28.

Embodiment 5 is an isolated monoclonal antibody or antigen-binding fragment thereof of any one of embodiments 1-3 that specifically binds to an epitope on an alpha-synuclein peptide comprising the amino acid sequence of SEQ ID NO: 31.

Embodiment 6 is an isolated monoclonal antibody or antigen-binding fragment thereof of any one of embodiments 1-5, wherein the monoclonal antibody or antigen-binding fragment thereof reduces the level of alpha-synuclein.

Embodiment 7 is an isolated monoclonal antibody or antigen-binding fragment thereof of any one of embodiments 1-6, wherein the monoclonal antibody or antigen-binding fragment thereof prevents or reduces the level of alpha-synuclein aggregation.

Embodiment 8 is a functional variant of the monoclonal antibody or antigen-binding fragment thereof of any one of embodiments 1-7.

Embodiment 9 is an immunoconjugate comprising the isolated monoclonal antibody or antigen-binding fragment thereof of any one of embodiments 1-7 and at least one therapeutic and/or detectable agent.

Embodiment 10 is an isolated nucleic acid encoding the monoclonal antibody or antigen-binding fragment thereof of any one of embodiments 1-7.

Embodiment 11 is a vector comprising the isolated nucleic acid of embodiment 10.

Embodiment 12 is a host cell comprising the vector of embodiment 11.

Embodiment 13 is a pharmaceutical composition, comprising the isolated monoclonal antibody or antigen-binding fragment thereof of any one of embodiments 1-7 and a pharmaceutically acceptable carrier.

Embodiment 14 is a method of preventing or reducing alpha-synuclein aggregation in a subject in need thereof, comprising administering to the subject the pharmaceutical composition of embodiment 13.

Embodiment 15 is a method of treating or preventing a disease characterized by Lewy bodies or alpha-synuclein aggregation in a subject in need thereof, comprising administering to the subject the pharmaceutical composition of embodiment 14.

Embodiment 16 is the method of embodiment 15, wherein the disease is selected from any synucleinopathy.

Embodiment 17 is the method of embodiment 15, wherein the disease is selected from the group consisting of Alzheimer's disease, Parkinson's disease, dementia with Lewy bodies, multiple system atrophy, and lysosomal-storage diseases.

Embodiment 18 is a method of producing the monoclonal antibody or antigen-binding fragment thereof of any one of embodiments 1-7, comprising culturing a cell comprising a nucleic acid encoding the monoclonal antibody or antigen-binding fragment under conditions to produce the monoclonal antibody or antigen-binding fragment, and recovering the monoclonal antibody or antigen-binding fragment from the cell or culture.

Embodiment 19 is a method of producing a pharmaceutical composition comprising the monoclonal antibody or antigen-binding fragment of any one of embodiments 1-7, comprising combining the monoclonal antibody or antigen-binding fragment with a pharmaceutically acceptable carrier to obtain the pharmaceutical composition.

Embodiment 20 is a method of determining a level of alpha-synuclein in a subject, the method comprising:
(a) obtaining a sample from the subject;
(b) contacting the sample with an isolated monoclonal antibody or antigen-binding fragment thereof of any one of embodiments 1-7; and
(c) determining the level of alpha-synuclein in the subject.

Embodiment 21 is the method of embodiment 20, wherein the sample is a tissue sample.

Embodiment 22 is the method of embodiment 21, wherein the tissue sample is a brain tissue sample.

Embodiment 23 is the method of embodiment 21, wherein the sample is a blood sample.

Embodiment 24 is a method of diagnosing a disease characterized by Lewy bodies or alpha-synuclein aggregation, comprising:
(a) obtaining a sample from the subject;
(b) contacting the sample with an isolated monoclonal antibody or antigen-binding fragment thereof of any one of embodiments 1-7; and
(c) detecting alpha-synuclein aggregates in the subject,
   wherein the detection of alpha-synuclein is diagnostic of the subject having a disease characterized by Lewy bodies or alpha-synuclein aggregates.

Embodiment 25 is a kit comprising at least one isolated monoclonal antibody or antigen-binding fragment thereof according to any one of embodiments 1-7.

### EXAMPLES

### Example 1: Generation of Alpha-Synuclein Constructs for Protein Production and Cell Assay

The full-length (140 amino acid) human α-synuclein gene SNCA was codon optimized for bacterial expression, synthesized, and subcloned into pUC57 vector at Genewiz, Inc. (Genewiz, Inc.; South Plainfield, N.J.) and a C-terminal AviTag, thrombin cleavage site, and his-tag were included in the synthesized gene. Xba1 and NotI sites were introduced by PCR using Phusion High Fidelity PCR Master Mix (Thermo Fisher; Waltham, Mass.), and the PCR products were double digested (New England Biolabs (NEB); Ipswich, Mass.), gel purified, and ligated into a pET28 vector following the manufacturer's protocol to generate His-Thrombin-Avi tagged full-length alpha-synuclein protein.

Human SNCA was PCR amplified from a pUC57 vector (Genewiz, Inc) with primers (Eton Bioscience, Inc.) overlapping with SNCA and pcDNA2004 vector and the 3′ primers have sequences encoding either −V5 and −HA sequence. The fragments were then gel purified and assembled into the vector using a Gibson Assembly Cloning Kit (NEB) following the manufacturer's protocol. The assembled mixture was transformed into DH5α competent cells (Thermo Fisher) and the correct plasmid was confirmed by sequencing. Similarly, to generate a positive control vector for cell assay, i.e. SNCA tagged with both V5- (C-terminus) and HA- (N-terminus), the fragment was generated by PCR amplifying the SNCA gene using a 5′ primer composed of nucleotides overlapping the pcDNA2004 vector −HA tag and 5′ of the SNCA gene and a 3′ primer composed of nucleotides overlapping the vector −V5 tag and 3′ of the SNCA gene. The PCR product was gel purified and assembled into the pcDNA2004 vector using Gibson Assembly Kit (NEB).

### Example 2: Recombinant Synuclein Purification and Biotinylation

Full length wild type (WT) α-synuclein, with a C-terminal avi-tag, thrombin cleavage site and his-tag, was produced by E. coli BL21 (DE3) (Thermo Fisher) cells in a 10 L wave bag. Two hours after induction with IPTG (Sigma-Aldrich; St. Louis, Mo.), the cells were harvested, and the pellets were stored at −80° C. The pellets were re-suspended and thawed in extraction buffer (BugBuster Master mix, EMD Millipore, Burlington, Mass.) supplemented with 1 tablet of protease inhibitor cocktail (Roche; Basel, Switzerland). The suspension was centrifuged, and the supernatant was heated for one hour at 60° C. and centrifuged at 5250×g at 4° C. for 30 min. The supernatant was buffer exchanged to 50 mM Bicine pH 8.3. Size exclusion chromatography with multi-angle static light scattering (SEC-MALS) analysis was used to estimate the total amount of α-synuclein protein. The required amounts of BirA enzyme, biotin, ATP and magnesium acetate were added for biotinylation overnight per manufacturer instructions (BirA biotin-protein ligase bulk reaction kit, Avidity LLC; Aurora, Colo.).

Biotinylation was confirmed by SEC-MALS analysis of biotin-synuclein binding to Streptavidin-PE. Then the biotinylated synuclein material was applied to His-tag purification resin, washed 3 times to remove impurities, and the α-synuclein was cleaved from the resin by thrombin during overnight incubation, followed by purification utilizing an SEC column.

The highly pure biotinylated α-synuclein was confirmed by SDS-PAGE and analytical SEC. In addition, purified and biotinylated α-synuclein was mixed with Streptavidin-PE and analyzed by SEC-MALS, showing that the α-synuclein was indeed biotinylated.

The reactivity of biotinylated α-synuclein was assessed by ELISA using a Streptavidin coated plate (see Example 5). The protein was fully reactive to antibodies of Syn303 (Biolegend; San Diego, Calif.) and C20 (Santa Cruz Biotechnology; Dallas, Tex.), which recognize the N-terminal (amino acids 1-5) and the C-terminal (amino acids 120-140) of synuclein, respectively.

### Example 3: Generation of α-Synuclein Baits and Single Cell Sorting of Bait-Specific Memory B Cells

To screen and clone naturally-occurring human mAbs to α-synuclein protein, a panel of 7 peptides covering the center region and C-terminus of α-synuclein (amino acids 61-140) were designed and synthesized. The panel included peptides phosphorylated at Ser-129 and Ser-87 and a truncation at amino acids 110 and 120. The peptides were synthesized by solid-phase chemistry with >95% purity confirmed by LC-MS (New England Peptide, Inc. and Eton Bioscience, Inc.). The Biotin and LC linker was synthesized at either the N- or C-terminus of the peptides as indicated. Alpha-synuclein peptide and protein sorting baits were prepared by mixing biotinylated peptides proteins with Streptavidin-APC or Streptavidin-PE (Thermo Fisher). The majority of peptides and free biotin (a negative control) were prepared at a 1:9 ratio (Streptavidin:peptide), incubated for 30 minutes on ice, and passed over a BioSpin 30 column (Bio-Rad Laboratories; Hercules, Calif.) to remove free peptide. The full-length protein and the aggregation prone C-terminally biotinylated peptide 61-95 were prepared at a 1:4 ratio and were used without column clean up. The ratio of peptides to Streptavidin was determined by SEC-MALS. Each tetramer was used at a final concentration of 36 nM, based on the Streptavidin concentration.

Whole blood (100 ml) from 25 clinically diagnosed Parkinson's Disease (PD) patients (age of 50-65) was purchased from Sanguine Biosciences (Sanguine Biosciences; Sherman Oaks, Calif.). Whole blood of 36 non-PD donors was obtained from The Scripps Research Institute (age of 21-68). Peripheral blood mononuclear cells (PBMCs) were isolated and cryopreserved as previously described (Pascual et al. (2017) Acta Neuropathol 133, 767-783). Briefly, the cells were isolated on Ficoll-Paque Plus (GE healthcare; Chicago, Ill.) and cryopreserved in 90% FBS and 10% DMSO. The procedure to screen memory B cells and recover monoclonal antibodies (mAbs) from donors was previously described (Pascual et al., 2017). Briefly, PBMCs from 3 healthy or 4-6 patient donors were thawed and rested overnight in complete RPMI media (RPMI with 10% FBS and 1% penicillin, 1% streptomycin) at 37° C. The B cells were enriched by positive selection with CD22+ magnetic beads (Miltenyi Biotec; Bergisch Gladbach; Germany). Cells were resuspended in FACS Buffer (Tris buffered saline (TBS) with 2 mM EDTA and 0.25% bovine serum albumin, Fraction V).

The extracellular markers IgG-FITC, CD19-PerCPCy5.5, and CD27-PECy7 (BD Biosciences; San Jose, Calif.) were added along with the PE and APC labelled protein and peptide panel. To determine nonspecific binding of the tetramers, an aliquot of antibody labeled cells was incubated with the biotin tetramers, used at the molar equivalent of the peptide pool. The cells and peptides were incubated for 1 hour at 4° C. with gentle mixing. After washing, the cells were re-suspended at 20×10⁶ cells/ml in FACS buffer. The live/dead marker DAPI (Thermo Fisher) was added prior to sorting on a Beckman Coulter MoFlo XDP or Astrios. The gates were set using the negative control as a guide to exclude nonspecific events. The CD19⁺, IgG⁺, CD27^{hi}, and antigen double-positive live cells were collected by single cell sorting. Cells were collected in cold real time PCR reaction buffer and RNaseOUT (Thermo Fisher). Plates were centrifuged briefly and stored at −80° C.

### Example 4: Recovery of Heavy and Light Chain Antibody Genes from Memory B Cells

Heavy and light chain cDNAs were then recovered by a two-step PCR approach from individual B-cells, and variable domain sequences were cloned and expressed in vitro as full-length recombinant IgG1 antibodies.

First-strand complementary DNA (cDNA) was generated from single sorted cells according to manufacturer's protocol (Superscript III, Invitrogen Corp.; Carlsbad, Calif.) with the following modifications: to each well containing a single B-cell, 0.5 μl of 10% NP-40, 1.0 μl of oligo dT, 1.0 μl of dNTP was added and samples were incubated at 65° C. for 5 minutes. After incubation, samples were placed on ice for 1 minute. The following was then added to each well: 2.0 μl of DTT, 4.0 μl of MgCl₂, 1.0 μl of SuperScript RT, and 0.5 μl of RNaseOut. Samples were incubated at 50° C. for 50 minutes, followed by incubation at 85° C. for 5 minutes. For the initial PCR (Step I), 2.5 μl of cDNA preparation was used as a template to amplify heavy and kappa or lambda light chains. Primer pools specific to the leader regions of antibody heavy, kappa light chain, and lambda light chain were used. A single reverse primer specific to the CH1 region, CK, and CL regions of the heavy, kappa light and lambda light chain, respectively, were used in the Step I PCR reaction.

For Step II, 2.5 μl of Step I PCR product was used as a template to amplify heavy, and kappa or lambda light chain variable regions. A pool of forward and reverse primers specifically designed to the framework 1 region of antibody heavy chain, kappa light chain, and lambda light chain were used to prepare DNA from the variable regions. Furthermore, Step II primers were designed to introduce XbaI and XhoI restriction sites for downstream cloning. Following the Step II amplification reactions, heavy and light chain variable domain PCR products were run on a 1% agarose gel. Heavy and light chain variable region fragments were purified according to the manufacturer's protocol (Qiagen; Hilden, Germany) and used in the Step III PCR reaction.

For Step III, the heavy and light chain variable region DNA fragments produced in Step II were linked into a single cassette via overlap extension PCR using: 1) a kappa linker or lambda linker (see linker preparation method below), which anneals to the 3′ end of the light chain Step II fragment and the 5′ end of the heavy chain Step II fragment, and contains either the kappa or lambda constant region, 2) a forward overlap primer containing an XbaI restriction site, and 3) a reverse primer containing an XhoI restriction site. This reaction results in an approximate 2400 bp or 2200 bp amplicon (i.e., cassette) for the kappa or lambda chains, respectively, consisting of the light chain variable region, linker, and heavy chain variable region. Following amplification, the overlap extension PCR reaction product was PCR-purified according to manufacturer's instructions (Qiagen PCR Purification Kit).

The linker fragment was amplified using pCB-IgG, a dual-CMV promoter vector generated in house and used to express both heavy and light chain genes. The linker fragment is 1765 or 1536 base pairs in length for kappa or lambda linker, respectively. The kappa linker contains from 5′ to 3′ an intron sequence followed by the kappa constant region, poly(A) termination sequence, and cytomegalovirus promoter sequence, allowing for one vector expression of the recombinant antibodies. The lambda linker contains the lambda constant region, poly(A) termination sequence, and cytomegalovirus promoter sequence. A common reverse primer and kappa-specific forward primer were used. The amplified fragment was separated on a 1% agarose gel and purified according to manufacturer's protocol (Qiagen Gel Extraction Kit).

Following purification of the overlap extension PCR product, the fragment was digested with XhoI and XbaI and subsequently separated on a 1% agarose gel. The band corresponding to the overlap cassette (˜2.4 kb) was purified and ligated into an IgG1 expression vector, pCB-IgG. Antibody variable genes were subcloned into this vector and antibodies were recombinantly expressed as IgG1 regardless of their original (native) isotype. All transformations were carried out using DH5a Max Efficiency cells (Invitrogen Corp.) and recovered in 250 μl of SOC for 1 hour at 37° C. Approximately 100 μl of recovered cells were plated onto a carbenicillin plate supplemented with 20 mM glucose. Plates were incubated overnight at 37° C. to allow for colony growth. The remaining recovered cell mixture was cultured with 4 ml of Super Broth (SB) media supplemented with 50 μg/ml carbenicillin and incubated overnight at 37° C. with shaking at 250 rpm. The following day, five colonies were picked per plate and grown in 3 ml of SB media supplemented with 50 μg/ml carbenicillin overnight at 37° C. Overnight cultures were used for DNA plasmid preparation (Qiagen).

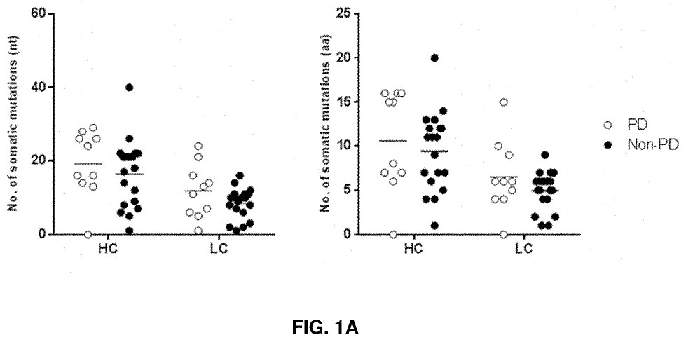

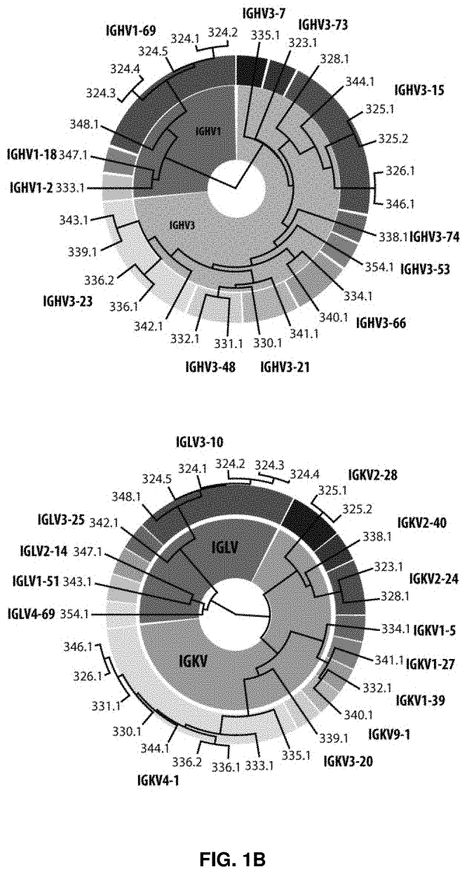

Sequence analysis was conducted on the heavy and light chain variable regions (FIG. 1A-1B). The number of somatic mutations away from germline sequences as determined by NCBI IgBlast was conducted at the amino acid (aa) and nucleotide (nt) level for both the heavy chain (HC) and light chain (LC) variable regions of antibodies (FIG. 1A). Phylogenetic analysis of recovered alpha-synuclein antibody heavy and light chain variable regions was conducted using the neighbor-joining algorithm (Jukes Cantor model; FIG. 1B). Heavy and light chain sequences are shown in Table 1 and Table 2. The complementarity determining region of the heavy and light chain variable regions are shown in Table 3 and Table 4.

TABLE 1
Sequences of heavy chain variable regions for anti-alpha-
synuclein mAbs

| mAb | VH |
|---|---|
| 323.1 | EVQLLESGGGLVQPGGSLKLSCAASGFTFSASAMHWVRQTSDKRLEWVGRIRN |
| | KANNYATAYAASVKGRFTISRDDSKNTAYLQMNSLKTEDTAVYYCTRHLSLGG |
| | NSVDYWGQGTPVTVSS (SEQ ID NO: 1) |
| 336.1 | EVQLVQSGGTLVQPGGSLRLSCAASGFTFSTYAISWVRQAPGRGLEWVSFITGD |
| | GSRILYADSVRGRFSISRDNSKNTLYLQMNSLRTDDTAMYYCVFNHYWGQGTL |
| | VTVSS (SEQ ID NO: 3) |
| 338.1 | QVQLVESGGDIVQPGGSLKLSCAASGFTFKSYWMHWVRQVPGKGLFWVSRINT |
| | FGNKTSYADSVRGRFSISRDNTKSILYLQMNSLKAEDTAVYYCARSTLGSFDYW |
| | GQGTLVTVSS (SEQ ID NO: 5) |

VH: heavy chain variable region

TABLE 2
Sequences of light chain variable regions for anti-alpha-
synuclein mAbs

| mAb | VL |
|---|---|
| 323.1 | DVVMTQSPLSSPVTLGQPASISCRASQSPVHSDGNTYLSWLQQRPGQPPRLLIYTI |
| | SNRFPGVPDRFSGSGAGTDFTLRISRVEAEDVGVYYCMQGTQFPYTFGQGTKLEI |
| | K (SEQ ID NO: 2) |
| 336.1 | AIQLTQSPDSLAVSLGERATINCKASQSLLYSSNNRNYLAWYQQKPGQPPKALIY |
| | WASTRESGVPDRFSGSGSGTDFTLTISSLQAEDVAVYYCHQYYTTHSFGQGTKL |
| | EIK (SEQ ID NO: 4) |
| 338.1 | AIQLTQSPDSLAVSLGERATINCKASQSLLYSSNNRNYLAWYQQKPGQPPKALIY |
| | WASTRESGVPDRFSGSGSGTDFTLTISSLQAEDVAVYYCHQYYTTHSFGQGTKL |
| | EIK (SEQ ID NO: 6) |

VL: light chain variable region

TABLE 3
CDR regions 1-3 of heavy chain for anti-alpha-synuclein mAbs

| | HC | | |
|---|---|---|---|
| | CDR1 | CDR2 | CDR3 |
| mAb | (SEQ ID NO:) | (SEQ ID NO:) | (SEQ ID NO:) |
| 323.1 | ASAMH (7) | RIRNKANNYATAYAASVKG (8) | HLSLGGNSVDY (9) |
| 336.1 | TYAIS (10) | FITGDGSRILYADSVRG (11) | NHY (12) |
| 338.1 | SYWMH (13) | RINTFGNKTSYADSVRG (14) | STLGSFDY (15) |

HC: heavy chain; CDR: complementarity determining region

TABLE 4
CDR regions 1-3 of heavy chain for anti-alpha-synuclein mAbs

| | LC | | |
|---|---|---|---|
| | CDR1 | CDR2 | CDR3 |
| mAb | (SEQ ID NO:) | (SEQ ID NO:) | (SEQ ID NO:) |
| 323.1 | RASQSPVHSDGNTYLS (16) | TISNRFP (17) | MQGTQFPYT (18) |
| 336.1 | KASQSLLYSSNNRNYLA (19) | WASTRES (20) | HQYYTTHS (21) |
| 338.1 | TSSQSLFDISDGNTYLD (22) | TLSYRAS (23) | MQRIESPST (24) |

LC: light chain; CDR: complementarity determining region

Cloned mAbs were transiently transfected in Expi293 cells (Thermo Fisher) and media was harvested by centrifugation at 72 hours post transfection. The IgG was purified from the culture media by Protein A affinity chromatography as previously described (Pascual et al., 2017). Media was passed twice through GE protein A Sepharose columns and washed with 50 ml of PBS. The media were eluted from the protein A affinity column in 100 mM sodium citrate buffer (pH 3.5) and neutralized with Tris buffer at pH 8.0. Eluates were dialyzed overnight against PBS then concentrated using an ultra-centrifugal unit (10,000 kDa CO; EMD Millipore). The IgGs were quantified using Protein A sensor tips on the Octet Red384 (ForteBio; Menlo Park, Calif.), and the quality of the IgGs was examined by SDS-PAGE under reducing and non-reducing conditions and size exclusion chromatography using FPLC AKTA Pure (GE Healthcare) to detect the presence of aggregates or degradation. The IgG monomer fraction was collected if an impurity was observed.

### Example 5: Screening and Confirmation of Reactivity of Anti-Synuclein mAbs to Synuclein Protein and Synuclein Peptides by ELISA

Pierce streptavidin-coated 96-well plates or Costar high binding plates were coated with the individual biotinylated synuclein peptides (400 nM final) or control (bovine actin, 1 ug/ml) diluted in TBS overnight at 4° C., respectively. The IgG concentration of the antibodies was determined by Octet with Protein A biosensors using a Protein A calibrator set (ForteBio). The anti-synuclein IgGs, diluted to 10 μg/ml in TBS-T (TBS containing 0.05% Tween 20 and 0.25% BSA), were added to the wells in duplicate and incubated at room temperature for 2 hours. After washing, goat-anti human IgG Fab-HRP (1:2000) or goat anti-mouse-HRP (1:4000, Jackson ImmunoResearch; West Grove, Pa.) were added and incubated for 1 hour. Plates were washed 5 times with TBS-T and developed with SureBlue Reserve TMB Microwell Peroxidase Substrate (KPL Inc.; Gaithersburg, Md.). The reaction was stopped by the addition of 100 μl of TMB Stop Solution (KPL), and the absorbance at 450 nm was measured using a Tecan M1000 plate reader. Antigen specific binding was defined as an OD450 greater than 0.5 and at least 3-fold above the secondary antibody alone. To confirm these results, the antibodies that met the criteria for antigen specificity were serially diluted 5-fold in TBS-T from a starting concentration of 10 ug/ml and retested against the antigen for which they demonstrated reactivity.

### Example 6: Qualitative Association and Dissociation Measurements by Octet Biolayer Interferometry to Full-Length Synuclein and Synuclein Peptides

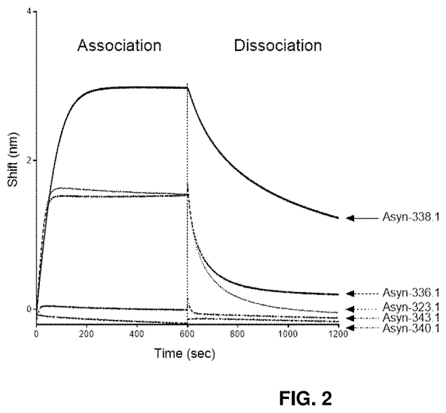

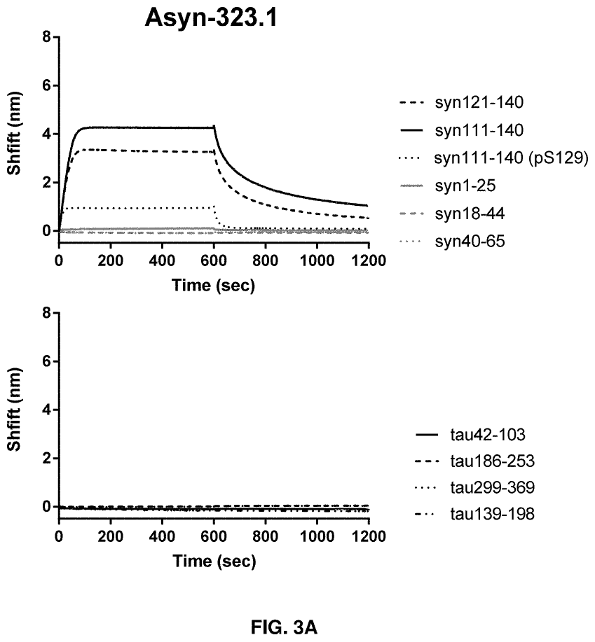

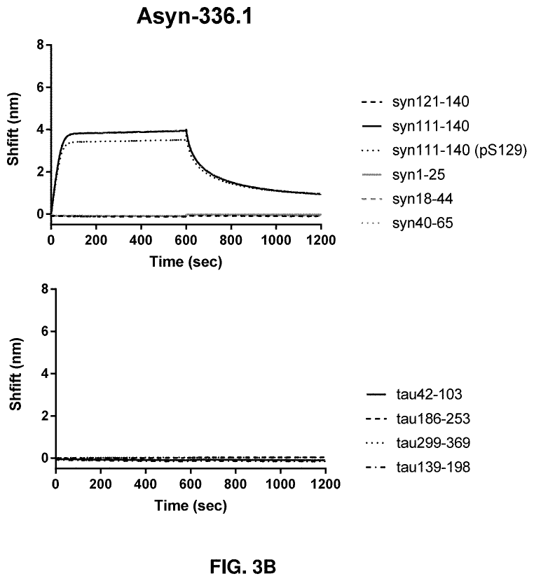

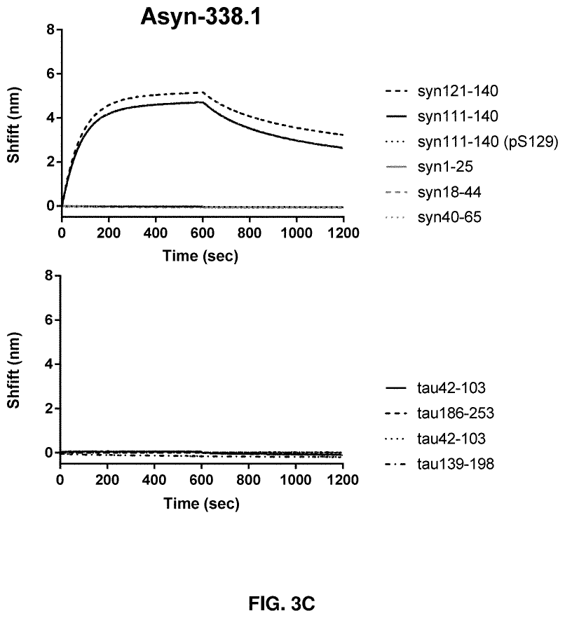

To assess the relative binding of the antibodies to full-length synuclein by biolayer interferometry (Octet Red 384; ForteBio), biotinylated synuclein protein was immobilized on Streptavidin (SA) Dip and Read biosensors for kinetics containing 10% ForteBio kinetics buffer as assay buffer. Real-time binding curves were measured by applying the sensor in a solution containing 100 nM antibody (FIG. 2). To induce dissociation, the biosensor containing the antibody-synuclein complex was immersed in assay buffer without antibody. Peptide epitope mapping was also assessed on Streptavidin (SA) Dip and Read biosensors using synuclein biotinylated peptides encompassing residues 1-25 (SEQ ID NO: 25), 18-44 (SEQ ID NO: 26), 40-65 (SEQ ID NO: 27), 121-140 (SEQ ID NO: 28), 111-140 (SEQ ID NO: 29), 111-140pS129 (SEQ ID NO: 30) (FIG. 3). All three antibodies bound to a peptide covering α-synuclein residues 111-140; however, only Asyn-323.1 and Asyn-338.1 bound peptide encompassing residues 121-140 (FIG. 3A and FIG. 3C). This suggests that Asyn-336.1 binds between residues 111-121 (FIG. 3B), while the other two antibodies bind more C-terminally, between residues 121 and 140. The three antibodies were tested for off-target binding to irrelevant tau peptides as shown in lower graph of each panel (FIG. 3). Sequences of the peptides that bound mAbs hantiASyn-323.1, hantiASyn-336.1 and hantiASyn-338.1 are shown in Table 5.

TABLE 5
Peptide regions bound by anti-synuclein mAbs

| Name | Residues | Epitope (SEQ ID NO:) |
|---|---|---|
| 323.1 | syn121-140 | DNEAYEMPSEEGYQDYEPEA (28) |
| 336.1 | syn111-121 | GILEDMPVDPD (31) |
| 338.1 | syn121-140 | DNEAYEMPSEEGYQDYEPEA (28) |

### Example 7: Immunoprecipitation Flow Cytometry (IP-FCM) Assay

Immunoprecipitation detected by flow cytometry (IP-FCM) is a sensitive method quantifying protein-protein interaction (Schrum et al. (2007) Sci STKE 2007, p12.). Synuclein tagged with −V5 and −HA was co-transfected into HEK293 cells. The antibody target −HA (or −V5) was conjugated to beads and used for immunoprecipitation of cell lysates, and then anti-V5-APC (or anti-HA-APC) antibody was introduced to the beads-antibody mixture. If intracellular synuclein aggregates resulting in both the −V5 and −HA tags being present within the aggregates, the positive signal can be detected and quantified using flow cytometry.

Antibody conjugation procedure was performed as described by Schrum et al (Schrum et al., 2007). Briefly, carboxyl groups on CML Latex Beads (Sigma-Aldrich) were activated with EDAC (50 mg/ml) dissolved in MES coupling Buffer (50 mM MES pH 6.0, 1 mM EDTA). Mouse monoclonal V5 antibody (Sigma-Aldrich) in PBS was added to the beads with shaking for 3-4 hours, and then the antibody was washed for later use.

Twenty-thousand HEK293 cells (ATCC; Manassas, Mass.; less than 30 passages) were plated in one well of a 96-well plate (Costar) in DMEM high glucose media (Cellgro) supplemented with 10% FBS (Gibco), 1% penicillin, 1% streptomycin and 1% L-glutamine (Hyclone) and incubated overnight at 37° C. in 8% CO2. The cells were transfected using FuGENE HD (Promega; Madison, Wis.). Briefly, 50 ng of Syn-HA and 50 ng Syn-V5 plasmids (or Syn-HA with negative control plasmid pcDNA-SNCA-Del61-92-V5) and 0.3 μl of FuGENE were mixed, incubated for 10 min, and then 10p of the transfection mixture were incubated with cells at 37° C. for 24 hours.

Monomeric α-synuclein (amino acids 1-140), generated as described above, was aggregated by incubation for 5 to 6 days at 37° C. in a rotator in the presence of small Teflon beads ( 1/16 inch in diameter). The samples were centrifuged for 15 min at 20,000 g to separate monomers/oligomers and aggregates. The pellet was stored at −80° C. to be used as bait and the supernatant was injected in SEC-MALS to quantify the α-synuclein content in the pellets. Synuclein aggregates were thawed at room temperature for 15 min, then vortexed and diluted to 1 mg/ml. 4 μg of aggregates and 200 μg of anti-synuclein antibody were mixed in a final volume of 50 μl of PBS, incubated for 2 hours with shaking at 37° C., and then diluted with 350 μl of media. The mixture was added to 4 wells with 100 μl in each well and incubated for 72 hours. Control antibodies used include, a positive control antibody, mouse Syn211 (ThermoFisher), mouse Isotype control, mouse anti-FLAG M2 (Sigma-Aldrich), and a human isotype control human (an anti-RSV antibody).

Cells were detached by adding 50 μl of 0.25% Trypsin-EDTA PBS (Gibco). Then 150 μl of media were added, and the cells were collected by centrifugation. The cells were lysed in 100p of ice-cold Lysis Buffer (1% Triton-X (Sigma-Aldrich) in TBS (Quality Biological; Gaithersburg, Md.) supplemented with 1× protease inhibitors (Roche) on ice. The lysates were centrifuged to remove cell debris (3000×g, 5 min, at 4° C.) and 80 ul of supernatant were transferred into a cold 96-well round bottom plate. 150,000 beads in 10 μl of Lysis Buffer was added to each well and incubated overnight at 4° C. with shaking at 750 rpm on a Microplate Genie (USA Scientific; Ocala, Fla.). Then the beads were centrifuged and washed twice in 200 ul ice-cold Post-IP Buffer (0.2% TritonX-100 in Lysis Buffer)

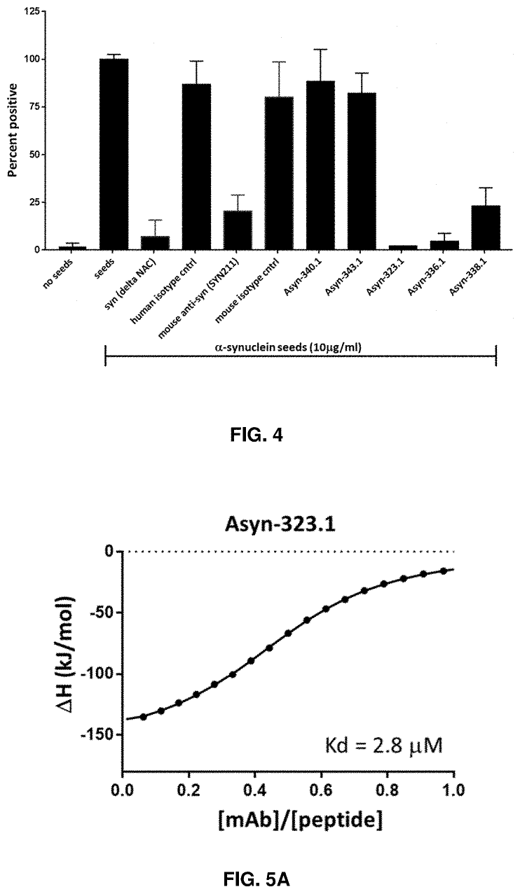

The capture beads were washed twice in ice cold FCM Staining Buffer (BD Biosciences) by centrifugation. Mouse anti-HA-SureLight APC antibody (Columbia Biosciences; Frederick, Md.) was added to each sample and incubated 40-60 min at 4° C. The samples were washed 3 times in FCM Staining Buffer, resuspended in 200 μl FCM staining buffer, and Flow Cytometry Analysis was performed using the MACSQuant (Miltenyi Biotec). The hantiAsyn 323.1, 336.1, and 338.1 monoclonal antibodies blocked intracellular alpha-synuclein aggregation (FIG. 4).

### Example 8: Affinity Measurements by Isothermal Titration Calorimetry (ITC)

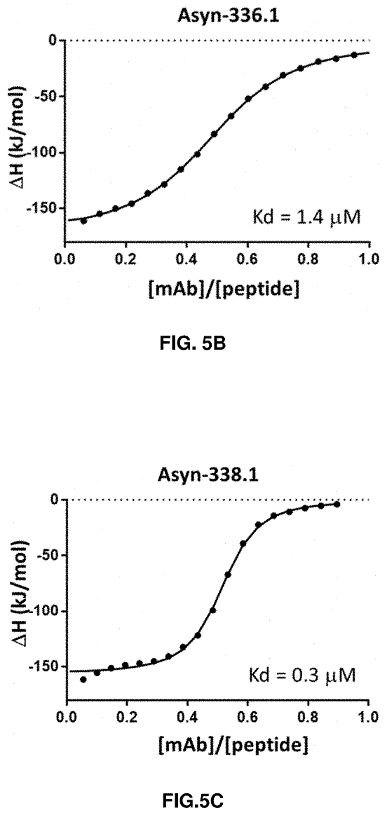

The affinities of antibodies for full length synuclein protein were determined in solution on a MicroCal Auto-iTC200 system (Malvern Panalystical; Malvern, United Kingdom). Synuclein peptides at 40 μM were titrated in 20 steps of 2 μl per step, in identical buffers containing 200 μM hantiASyn-323.1, hantiASyn-336.1 and hantiASyn-338.1, respectively. The thermodynamic parameters and the equilibrium dissociation constants, Kd, were determined upon fitting the ITC data to a model assuming a single set of binding sites corresponding to an antibody:synuclein (1:2) binding model (FIG. 5).

### Example 9: Immunohistochemistry on Post Mortem Human Brain Tissue

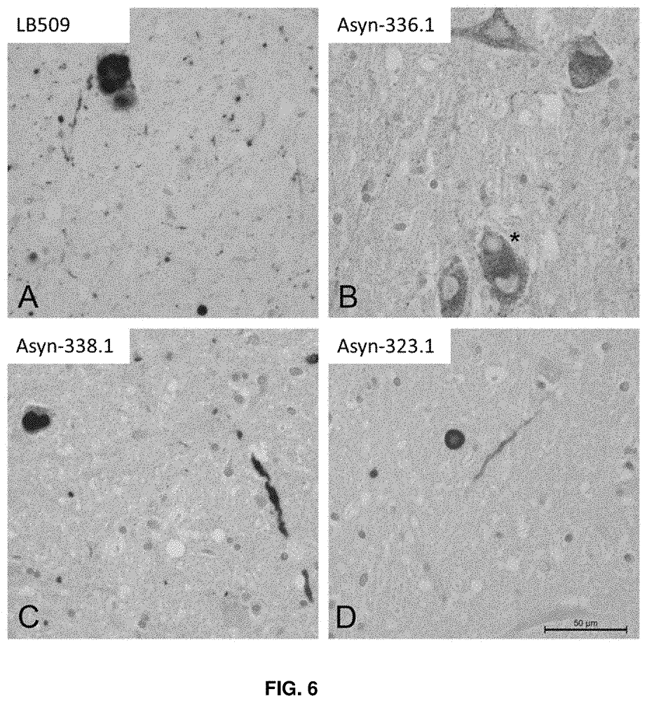

Post-mortem human brain tissue was obtained from the Vrije Universiteit Medical Center, Amsterdam, Netherlands. Sections (5 μm-thick) from formalin-fixed paraffin embedded PD brain tissue (mesencephalon) were mounted on coated glass slides (Menzel glaser superfrost plus, VWR International; Leuven, Belgium) and dried overnight at 37° C. Slides were deparaffinized in xylene and rehydrated through descending alcohol concentrations. Endogenous peroxidase activity was blocked by incubating the slides for 30 min in phosphate buffered saline (PBS; pH7.4) containing 0.3% H₂O₂. Between incubation steps, the sections were rinsed in PBS. All antibodies were diluted in antibody diluent (Immunologic; Duiven, Netherlands) and incubated overnight at room temperature. Human anti-alpha-synuclein antibodies hantiAsyn-323.1, hantiAsyn-336.1, hantiAsyn-338.1 were used at a concentration of 0.5 μg/ml. Mouse anti-alpha-synuclein antibody LB509 (Thermofisher) was used at a concentration of 1.25 μg/ml. Primary antibodies were detected with goat-anti-Human-HRP (dilution 1:250, 60 min at room temperature, Santa Cruz) or goat-anti-mouse/rabbit-HRP (ready-to-use, 30 min at room temperature; EnVision Dako; Glostrup, Denmark). To visualise the staining 3,3′-diaminobenzidine (DAB; Dako; Glostrup, Denmark) was used. Slides were counterstained with haematoxylin, dehydrated and mounted with Quick-D mounting medium (Klinipath; Duiven, Netherlands) (FIG. 6).

### Example 10: Generation of De-Risked and Fc Engineered Anti-Synuclein Monoclonal Antibodies

The heavy and light chain variable regions (VH and VL) for each anti-synuclein antibody clone isolated in Example 4 are analyzed for the presence of free cysteines and potential sites prone to post-translational modifications, including glycosylation, oxidation and deamidation sites. Non-conserved cysteines in the variable regions are mutated to serine and amino acid mutations consisting of structurally conserved and/or germline-based substitutions are used to remove these sites. For glycosylation sites, several mutations can be used, including replacement of asparagine for the conservative glutamine or mutations to germline encoded residues. Modifications to the deamidation sites include replacement of aspartic acid for asparagine and serine or alanine for glycine. Sites of potential oxidation are not modified. To increase the binding affinity to FcRn and thus increase the half-life of IgG1 mAbs in vivo, several mutations located at the boundary between the CH2 and CH3 region are generated, including M252Y/S254T/T256E plus H433K/N434F (Vaccaro et al. (2005) Nat Biotechnol. 23(10):1283-8) or T250Q/M428L (Hinton et al. (2004) J Biol Chem. 279(8):6213-6) mutations, all of which have been shown to increase IgG1 binding to FcRn. All substitutions are generated by site-directed mutagenesis per manufacturer's instructions (QuickChange II, Agilent Technologies; Santa Clara, Calif.). Nucleotide sequences for all constructs are verified according to standard techniques known to the skilled artisan.

### Example 11: Alanine Scanning Mutagenesis to Identify Contact Residues in Anti-Synuclein Antibody Epitopes

To assess the specificity and amino acid contribution to binding of each of the recovered mAbs to synuclein, their reactivity to synuclein peptides with each position replaced with alanine is tested by ELISA. Biotinylated synuclein peptides are synthesized commercially and dissolved in water at 1 mg/ml and frozen at −80° C. Briefly, 96-well streptavidin binding plates (Thermo Fisher) are coated with 2 μg/ml of synuclein peptides diluted in TBS and incubated overnight at 4° C. The following day, plates are washed with TBS-T and subsequently blocked with 2.5% BSA in TBS for 2 hr at room temperature. Following blocking, purified anti-syn IgGs (i.e., Asyn-323.1, Asyn-336.1, and Asyn-338.1) are diluted to 5 μg/ml and titrated 5-fold in TBS plus 0.25% BSA and incubated at room temperature for 2 hr. Plates are washed 5-times with TBS-T followed by the addition of secondary antibody [goat Anti-Human IgG F(ab′)₂ (Jackson Labs) at 1:2000 dilution], diluted in TBS plus 0.25% BSA, and incubated at room temperature for 1 hr. Following incubation, plates are washed 4-times in TBS-T and developed with SureBlue Reserve TMB Microwell Peroxidase Substrate (KPL) for approximately 90 sec. The reaction is immediately halted by the addition of TMB Stop Solution (KPL) and the absorbance at 450 nm is measured using an ELISA plate reader. Each experiment is conducted in triplicate and reactivity is considered positive when values are equal to or higher than an OD of 0.3 in the ELISA assay. Antibody reactivity is scored as no binding (−), weak (−/+), moderate (+), or strong (++). (−) for average of two O.D.450 nm readings <0.3; (−/+) for >0.5 and <1.0; (+) for >1.0 and <1.5; (++) for >1.5.

### Example 12: Affinity Maturation of Anti-Synuclein Antibodies

The coding sequence for scFv corresponding to Asyn-323.1, Asyn-336.1, and Asyn-338.1 is cloned into an inducible prokaryotic expression vector containing the phage M13 pIII gene. In this Example, random mutations are deliberately introduced in the scFv by error prone PCR (Genemorph II EZClone Domain Mutagenesis kit, Agilent technologies) after which the DNA is transformed into TG1 bacteria. The transformants are grown to mid-log phase and infected with helper phages that provide all the genes required for phage assembly. ScFv expressing phages are rescued by a CT helper phage genome, lacking the infectivity domains N1 and N2 of protein pIII and thus rendering phage particles that are only infective if they display the scFv linked to the full length pIII (Kramer et al. (2003) Nucleic Acids Res. 31(11): e59.). Phage libraries are screened using magnetic beads coated with full-length synuclein and/or cognate Asyn peptides in immunotubes. To deselect nonspecific binders, the tubes are coated with a non-relevant peptide lacking the Asyn mAb epitope. To ensure maturation against the correct epitope, selection is continued using beads coated with the cognate peptide. Eluted phages are used to infect XL1-blue F′ bacteria which were cultured and infected with helper phages to rescue phages used for subsequent selection rounds. After three rounds of panning, individual phage clones are isolated and screened in phage ELISA for binding to full-length synuclein and/or cognate Asyn-323.1, Asyn-336.1, and Asyn-338.1 peptides. Selected variant clones are converted and expressed as IgG1 to further assess their affinity in solution by Octet and isothermal calorimetry.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the present description.

1. An isolated monoclonal antibody or antigen-binding fragment thereof comprising a heavy chain complementarity determining region 1 (HCDR1), a heavy chain complementarity determining region 2 (HCDR2), a heavy chain complementarity determining region 3 (HCDR3), a light chain complementarity determining region 1 (LCDR1), a light chain complementarity determining region 2 (LCDR2), and a light chain complementarity determining region 3 (LCDR3), having the polypeptide sequences of:
(a) SEQ ID NOs: 7, 8, 9, 16, 17, and 18, respectively;
(b) SEQ ID NOs: 10, 11, 12, 19, 20, and 21, respectively; or
(c) SEQ ID NOs: 13, 14, 15, 22, 23, and 24, respectively;
wherein the antibody or antigen-binding fragment thereof specifically binds alpha-synuclein.

2. The isolated monoclonal antibody or antigen-binding fragment thereof of claim 1, comprising at least one of:
a heavy chain variable region having a polypeptide sequence at least 95% identical to a sequence selected from the group consisting of SEQ ID NO: 1, SEQ ID NO: 3, and SEQ ID NO: 5, and
a light chain variable region having a polypeptide sequence at least 95% identical to a sequence selected from the group consisting of SEQ ID NO: 2, SEQ ID NO: 4, and SE ID NO: 6.

3. The isolated monoclonal antibody or antigen-binding fragment thereof of claim 2, comprising:
(a) a heavy chain variable region having the polypeptide sequence of SEQ ID NO:1, and a light chain variable region having the polypeptide sequence of SEQ ID NO:2;
(b) a heavy chain variable region having the polypeptide sequence of SEQ ID NO:3, and a light chain variable region having the polypeptide sequence of SEQ ID NO:4; or
(c) a heavy chain variable region having the polypeptide sequence of SEQ ID NO:5, and a light chain variable region having the polypeptide sequence of SEQ ID NO:6.

4. The isolated monoclonal antibody or antigen-binding fragment thereof of claim 1 that specifically binds to an epitope on an alpha-synuclein peptide comprising the amino acid sequence of SEQ ID NO: 28.

5. The isolated monoclonal antibody or antigen-binding fragment thereof of claim 1 that specifically binds to an epitope on an alpha-synuclein peptide comprising the amino acid sequence of SEQ ID NO: 31.

6. The isolated monoclonal antibody or antigen-binding fragment thereof of claim 1, wherein the monoclonal antibody or antigen-binding fragment thereof reduces the level of alpha-synuclein.

7. The isolated monoclonal antibody or antigen-binding fragment thereof of claim 1, wherein the monoclonal antibody or antigen-binding fragment thereof prevents or reduces the level of alpha-synuclein aggregation.

8. A functional variant of the monoclonal antibody or antigen-binding fragment thereof of claim 1.

9. An immunoconjugate comprising:
the isolated monoclonal antibody or antigen-binding fragment thereof of claim 1, and
at least one therapeutic and/or detectable agent.

10. An isolated nucleic acid encoding the monoclonal antibody or antigen-binding fragment thereof of claim 1.

11. A vector comprising the isolated nucleic acid of claim 10.

12. A host cell comprising the vector of claim 11.

13. A pharmaceutical composition, comprising:
the isolated monoclonal antibody or antigen-binding fragment thereof of claim 1, and
a pharmaceutically acceptable carrier.

14. A method of preventing or reducing alpha-synuclein aggregation in a subject in need thereof, comprising administering to the subject the pharmaceutical composition of claim 13.

15. A method of treating or preventing a disease characterized by Lewy bodies or alpha-synuclein aggregation in a subject in need thereof, comprising administering to the subject the pharmaceutical composition of claim 13.

16. The method of claim 15, wherein the disease is a synucleinopathy.

17. The method of claim 15, wherein the disease is selected from the group consisting of Alzheimer's disease, Parkinson's disease, dementia with Lewy bodies, multiple system atrophy, and lysosomal-storage diseases.

18. A method of producing the monoclonal antibody or antigen-binding fragment thereof of claim 1, comprising culturing a cell comprising a nucleic acid encoding the monoclonal antibody or antigen-binding fragment under conditions to produce the monoclonal antibody or antigen-binding fragment, and recovering the monoclonal antibody or antigen-binding fragment from the cell or culture.

19. A method of producing a pharmaceutical composition comprising the monoclonal antibody or antigen-binding fragment of claim 1, comprising combining the monoclonal antibody or antigen-binding fragment with a pharmaceutically acceptable carrier to obtain the pharmaceutical composition.

20. A method of determining a level of alpha-synuclein in a subject, the method comprising:
(a) obtaining a sample from the subject;
(b) contacting the sample with an isolated monoclonal antibody or antigen-binding fragment thereof of claim 1; and
(c) determining the level of alpha-synuclein in the subject.

21. The method of claim 20, wherein the sample is a tissue sample.

22. The method of claim 21, wherein the tissue sample is a brain tissue sample.

23. The method of claim 21, wherein the sample is a blood sample.

24. A method of diagnosing a disease characterized by Lewy bodies or alpha-synuclein aggregation, comprising:
(a) obtaining a sample from the subject;
(b) contacting the sample with an isolated monoclonal antibody or antigen-binding fragment thereof of claim 1; and
(c) detecting alpha-synuclein aggregates in the subject,
wherein the detection of alpha-synuclein is diagnostic of the subject having a disease characterized by Lewy bodies or alpha-synuclein aggregates.

25. A kit comprising at least one isolated monoclonal antibody or antigen-binding fragment thereof according to claim 1.


SEQUENCE LISTING (31 sequences)

SEQ ID NO: 1 - protein, 122 aa, Artificial Sequence
  323.1 heavy chain variable region
EVQLLESGGGLVQPGGSLKLSCAASGFTFSASAMHWVRQTSDKRLEWVGRIRNKANNYATAYAASVKGRFTISRDDSKNTAYLQMNSLKTEDTAVYYCTRHLSLGGNSVDYWGQGTPVTVSS

SEQ ID NO: 2 - protein, 112 aa, Artificial Sequence
  323.1 light chain variable region
DVVMTQSPLSSPVTLGQPASISCRASQSPVHSDGNTYLSWLQQRPGQPPRLLIYTISNRFPGVPDRFSGSGAGTDFTLRISRVEAEDVGVYYCMQGTQFPYTFGQGTKLEIK

SEQ ID NO: 3 - protein, 112 aa, Artificial Sequence
  336.1 heavy chain variable region
EVQLVQSGGTLVQPGGSLRLSCAASGFTFSTYAISWVRQAPGRGLEWVSFITGDGSRILYADSVRGRFSISRDNSKNTLYLQMNSLRTDDTAMYYCVFNHYWGQGTLVTVSS

SEQ ID NO: 4 - protein, 112 aa, Artificial Sequence
  336.1 light chain variable region
AIQLTQSPDSLAVSLGERATINCKASQSLLYSSNNRNYLAWYQQKPGQPPKALIYWASTRESGVPDRFSGSGSGTDFTLTISSLQAEDVAVYYCHQYYTTHSFGQGTKLEIK

SEQ ID NO: 5 - protein, 117 aa, Artificial Sequence
  338.1 heavy chain variable region
QVQLVESGGDIVQPGGSLKLSCAASGFTFKSYWMHWVRQVPGKGLFWVSRINTFGNKTSYADSVRGRFSISRDNTKSILYLQMNSLKAEDTAVYYCARSTLGSFDYWGQGTLVTVSS

SEQ ID NO: 6 - protein, 112 aa, Artificial Sequence; identical to SEQ ID NO: 4
  338.1 light chain variable region

SEQ ID NO: 7 - protein, 5 aa, Artificial Sequence
  323.1 heavy chain CDR1
ASAMH

SEQ ID NO: 8 - protein, 19 aa, Artificial Sequence
  323.1 heavy chain CDR2
RIRNKANNYATAYAASVKG

SEQ ID NO: 9 - protein, 11 aa, Artificial Sequence
  323.1 heavy cain CDR3
HLSLGGNSVDY

SEQ ID NO: 10 - protein, 5 aa, Artificial Sequence
  336.1 heavy chain CDR1
TYAIS

SEQ ID NO: 11 - protein, 17 aa, Artificial Sequence
  336.1 heavy chain CDR2
FITGDGSRILYADSVRG

SEQ ID NO: 12 - protein, 3 aa, Artificial Sequence
  336.1 heavy chain CDR3
NHY

SEQ ID NO: 13 - protein, 5 aa, Artificial Sequence
  338.1 heavy chain CDR1
SYWMH

SEQ ID NO: 14 - protein, 17 aa, Artificial Sequence
  338.1 heavy chain CDR2
RINTFGNKTSYADSVRG

SEQ ID NO: 15 - protein, 8 aa, Artificial Sequence
  338.1 heavy chain CDR3
STLGSFDY

SEQ ID NO: 16 - protein, 16 aa, Artificial Sequence
  323.1 light chain CDR1
RASQSPVHSDGNTYLS

SEQ ID NO: 17 - protein, 7 aa, Artificial Sequence
  323.1 light chain CDR2
TISNRFP

SEQ ID NO: 18 - protein, 9 aa, Artificial Sequence
  323.1 light chain CDR3
MQGTQFPYT

SEQ ID NO: 19 - protein, 17 aa, Artificial Sequence
  336.1 light chain CDR1
KASQSLLYSSNNRNYLA

SEQ ID NO: 20 - protein, 7 aa, Artificial Sequence
  336.1 light chain CDR2
WASTRES

SEQ ID NO: 21 - protein, 8 aa, Artificial Sequence
  336.1 light chain CDR3
HQYYTTHS

SEQ ID NO: 22 - protein, 17 aa, Artificial Sequence
  338.1 light chain CDR1
TSSQSLFDISDGNTYLD

SEQ ID NO: 23 - protein, 7 aa, Artificial Sequence
  338.1 light chain CDR2
TLSYRAS

SEQ ID NO: 24 - protein, 9 aa, Artificial Sequence
  338.1 light chain CDR3
MQRIESPST

SEQ ID NO: 25 - protein, 25 aa, Artificial Sequence
  synuclein 1-25
MDVFMKGLSKAKEGVVAAAEKTKQG

SEQ ID NO: 26 - protein, 27 aa, Artificial Sequence
  synuclein 18-44
AAEKTKQGVAEAAGKTKEGVLYVGSKT

SEQ ID NO: 27 - protein, 26 aa, Artificial Sequence
  synuclein 40-65
VGSKTKEGVVHGVATVAEKTKEQVTN

SEQ ID NO: 28 - protein, 20 aa, Artificial Sequence
  synuclein 121-140
DNEAYEMPSEEGYQDYEPEA

SEQ ID NO: 29 - protein, 30 aa, Artificial Sequence
  synuclein 111-140
GILEDMPVDPDNEAYEMPSEEGYQDYEPEA

SEQ ID NO: 30 - protein, 30 aa, Artificial Sequence; identical to SEQ ID NO: 29
  synuclein 111-140 (pS129)
  MOD_RES (19)..(19): PHOSPHORYLATION

SEQ ID NO: 31 - protein, 11 aa, Artificial Sequence
  synuclein 111-121
GILEDMPVDPD